(19) United States
(12) Patent Application Publication
(10) Pub. No.: US 2026/0083544 A1
(43) Pub. Date: Mar. 26, 2026

(54) STENT GRAFT WITH LOW PROFILE PASSIVE SEALING SKIRT AND METHOD

(71) Applicant: Medtronic Vascular, Inc., Santa Rosa, CA (US)

(72) Inventors: Adam J. SHIPLEY, San Rafael, CA (US); Torey HOVEST, Copley, OH (US); Caitlin M. OWENSON, Santa Rosa, CA (US); Anish Sanjay NIGADE, Santa Rosa, CA (US); Steven Michael CLAESSENS, Tualatin, OR (US); Erik C. GRISWOLD, Penngrove, CA (US); Lijin Rajendran NANU, Santa Rosa, CA (US); Keith D. PERKINS, Santa Rosa, CA (US)

(21) Appl. No.: 19/106,750

(22) PCT Filed: Sep. 15, 2023
(86) PCT No.: PCT/US2023/032898

§ 371 Date: Feb. 26, 2025

Related U.S. Application Data
(60) Provisional application No. 63/407,001, filed on Sep. 15, 2022.
(60) Provisional application No. 63/538,462, filed on Sep. 14, 2023.
(60) Provisional application No. 63/538,460, filed on Sep. 14, 2023.

(51) Int. Cl. A61F 2/07

(52) U.S. Cl. CPC A61F 2/07; A61F 2002/075; A61F 2220/0075; A61F 2250/0069

(57) ABSTRACT

The techniques of this disclosure generally relate to a method including providing a sealing stent graft. The sealing stent graft includes a graft material, a stent coupled to the graft material, a sealing skirt of the graft material lying flat on the stent, and a collapsing suture coupled to the graft material. The method further includes expanding the stent to cause the collapsing suture to collapse the sealing skirt. Accordingly, the sealing skirt lies flat and does not impact the packing density of sealing stent graft when in a delivery configuration. During deployment, the collapse of the sealing skirt causes the sealing skirt to protrude radially outward. This thickening of the sealing skirt improves sealing of the sealing stent graft with other structures, e.g., vessels.

### CROSS-REFERENCE TO RELATED APPLICATIONS

This application claims the benefit of U.S. provisional application Ser. No. 63/407,001 filed Sep. 15, 2022, and U.S. provisional application Ser. No. 63/538,460 filed Sep. 14, 2023, and U.S. provisional application Ser. No. 63/538,462 filed Sep. 14, 2023, the disclosures of which is hereby incorporated in their entirety by reference.

### TECHNICAL FIELD

The present application is generally related to an intra-vascular device and method. For example, the present application relates to a device for treatment of intra-vascular diseases. The present application also is generally related to heart implant devices (e.g., transcatheter aortic valve replacement (TAVR) device) having flippable sealing skirts or having expanding, axially pleating sealing skirts.

### BACKGROUND

A conventional stent graft typically includes a radially expandable reinforcement structure, formed from a plurality of annular stent rings, and a cylindrically shaped layer of graft material defining a lumen to which the stent rings are coupled. Stent grafts are well known for use in tubular shaped human vessels.

To illustrate, endovascular aneurysmal exclusion is a method of using a stent graft to exclude pressurized fluid flow from the interior of an aneurysm, thereby reducing the risk of rupture of the aneurysm and the associated invasive surgical intervention. However, challenges exist in certain anatomies to provide an adequate seal between the stent graft and the vessel to effectively exclude the aneurysm from the pressurized fluid flow.

Diseased or otherwise deficient heart valves may be repaired or replaced using a variety of heart valve surgeries. One type of heart valve surgery is an open-heart surgical procedure conducted under general anesthesia. During an open-heart surgical procedure, pumping of the heart is stopped and blood flow is controlled by a heart-lung bypass machine. Due to their highly invasive nature, open-heart surgical procedures expose a patient to one or more potential, serious risks (e.g., infection, stroke, renal failure, and adverse effects of the heart-lung machine).

In light of the invasiveness and attendant serious risks of open-heart surgical procedures, less invasive percutaneous replacement of heart valves has been proposed and implemented. One type of such procedure is referred to as a transcatheter heart valve procedure in which a valve assembly is inserted and delivered into the heart via a delivery system (e.g., a catheter) through a relatively small opening in the skin to access the vasculature of a patient.

Prosthetic heart valve designs aim to mimic the function of natural heart valve designs. One type of prosthetic heart valve design is referred to a self-expanding valve (e.g., a supra-annular design). The self-expanding valve design includes an expandable stent frame formed of a self-expanding material (e.g., a nickel titanium alloy or Nitinol). Leaflets may be mounted onto the expandable stent frame to replicate a natural heart valve. A wrap may be sewn onto the expandable stent frame to reduce paravalvular leak (PVL). PVL is a leak caused by a gap between natural heart tissue and the replacement valve replacement.

While the wrap may reduce or resist PVL, the wrap may increase the profile of the self-expanding valve in the delivery position (e.g., compressed position). This increased profile may present challenges to delivery of the self-expanding valve to a deployment location (e.g., an aortic valve location) for deployment into a deployed position.

### SUMMARY

The techniques of this disclosure generally relate to a method including providing a sealing stent graft. The sealing stent graft includes a graft material, a stent coupled to the graft material, a sealing skirt of the graft material lying flat on the stent, and a collapsing suture coupled to the graft material. The method further includes expanding the stent to cause the collapsing suture to collapse the sealing skirt. In one or more embodiments, the sealing skirt lies flat and does not impact the packing density of sealing stent graft when in a delivery configuration. During deployment, the collapse of the sealing skirt causes the sealing skirt to protrude radially outward. This thickening of the sealing skirt improves sealing of the sealing stent graft with other structures, e.g., vessels. The collapse of the sealing skirt may create ruffles in the sealing skirt. The method may further include positioning the sealing stent graft in a vessel prior to the expanding step and deploying the sealing stent graft in the vessel. In one or more embodiments, the sealing skirt creates a seal with the vessel. The method may further include creating a seal with the sealing skirt between the sealing stent graft and a first component.

In one aspect, the present disclosure provides an assembly including a sealing stent graft. The sealing stent graft includes a graft material, a stent coupled to the graft material, and a collapsing suture coupled to the graft material. When the sealing stent graft is in a delivery configuration, a sealing skirt of the graft material lies flat on the stent. When the sealing stent graft is in a deployed configuration, the sealing skirt is collapsed by the collapsing suture and protrudes from the stent. The graft material may further include an exclusion section. The stent may further include first crowns, second crowns, and structs extending between the first crowns and the second crowns. The second crowns may be attached to the graft material at anchor points. The sealing skirt and the exclusion section may meet at the anchor points. The graft material may include an edge defining an opening and overlapping the struts. The stent may extend from the second crowns past the edge to the first crowns.

In another aspect, the present disclosure provides an assembly including a graft material, a stent, and a collapsing suture. The graft material has an edge. The stent is coupled to the graft material at anchor points. The graft material has a sealing skirt between the anchor points and the edge. The collapsing suture is coupled to the sealing skirt and to the graft material at periodic points in an alternating repeating pattern. The periodic points may include first periodic points that define a first suture line and second periodic points that define a second suture line. Upon expansion of the stent, a longitudinal distance between the first suture line and the second suture line may decrease. Upon expansion of the stent, a circumferential distance between the first suture points may increase and a circumferential distance between the second suture points may increase. Upon expansion of the stent, the collapsing suture may collapse the sealing skirt. The sealing skirt may protrude radially outward when collapsed. The assembly may further include a sealing stent graft that includes the graft material, the stent, and the collapsing suture, and a first component. The sealing skirt may be located within the first component. Alternatively, the first component may be located within the sealing skirt. The sealing skirt may protrude radially inward when collapsed. The sealing skirt may lay flat upon the stent prior to expansion of the stent.

In one embodiment, a heart implant device is disclosed. The heart implant device includes a frame extending along a longitudinal axis and having an inflow section, an outflow section, and a constricted section extending between the inflow section and the outflow section. The heart implant device further includes a skirt anchored to the inflow section of the frame. The heart implant device also includes a collapsing suture anchored to the inflow section of the frame and the skirt. The collapsing suture has a repeating shape having a delivery amplitude in a delivery state of the frame and a deployed amplitude in a deployed state of the frame. A reduced amplitude from the delivery amplitude to the deployed amplitude creates a force causing a portion of the skirt to outwardly rotate at a hinge line to form an overlap between the portion of the skirt and a portion of the inflow section of the frame. The overlap is configured to enhance sealing of the heart implant device and/or reduce paravalvular leak (PVL) in the deployed state.

The skirt may be anchored to the inflow section of the frame at skirt/frame anchor points forming a skirt/frame anchor line. The collapsing suture may be anchored to the inflow section of the frame at suture/frame anchor points forming a suture/frame anchor line. The collapsing suture may be anchored to the skirt at suture/skirt anchor points forming a suture/skirt anchor line. The skirt/frame anchor line may be longitudinally located between the suture/frame anchor line and the suture/skirt anchor line. The skirt/frame anchor points may be offset the suture/frame anchor points and the suture/skirt anchor points. The frame may have inner and outer surfaces and the skirt may have inner and outer surfaces. The suture/frame anchor points may anchor to the outer surface of the frame. The suture/skirt anchor points may anchor to the outer surface of the skirt. The outer surface of the frame and the outer surface of the skirt may contact in the deployed state. The hinge line may be spaced apart the skirt/frame anchor line toward the suture/skirt anchor line. The repeating shape may be a sinusoidal shape. The force includes a radial component. The skirt may include first and second sections. The first and second sections of the skirt may align with the inflow section of the frame in the delivery state. The first section of the skirt may align with the inflow section in the deployed state. The second section of the skirt may contact the inflow section of the frame in the deployed state.

Another embodiment discloses a heart implant device including a frame extending along a longitudinal axis and having an inflow section, an outflow section, and a constricted section extending between the inflow section and the outflow section. The heart implant device further includes a skirt having first and second end sections. The first end section of the skirt is anchored to the inflow section of the frame at skirt/frame anchor points forming a skirt/frame anchor line. The heart implant device also includes a collapsing suture anchored to the inflow section of the frame at suture/frame anchor points forming a suture/frame anchor line and the second end section of the skirt at suture/skirt anchor points forming a suture/skirt anchor line. The collapsing suture has a repeating shape having a delivery amplitude in a delivery state of the heart implant device and a deployed amplitude in a deployed state of the heart implant device. A reduced amplitude from the delivery amplitude to the deployed amplitude creates a force causing the skirt to flip about the skirt/frame anchor line and to form a sealing portion of the skirt.

The sealing portion of the skirt may be pleated in the deployed state. The collapsing suture may include a first set of legs extending in a first direction and a second set of legs extending in a second direction intersecting the first direction to form a scissor arm. The suture/skirt anchor line may be longitudinally located between the outflow section of the frame and the skirt/frame anchor line in the delivery state and between the skirt/frame anchor line and the suture/frame anchor line in the deployed state. A portion of the skirt longitudinally may align with the constricted section of the frame in the delivery state.

Yet another embodiment discloses a heart implant device including a frame extending along a longitudinal axis and having an inflow section, an outflow section, and a constricted section extending between the inflow section and the outflow section. The heart implant device also includes a skirt anchored to the inflow section of the inflow section of the frame at first skirt/frame anchor points forming a first skirt/frame anchor line and at second skirt/frame anchor points forming a second skirt/frame anchor line. The heart implant device also includes a collapsing suture anchored to the inflow section of the frame at suture/frame anchor points forming a suture/frame anchor line and the skirt at suture/skirt anchor line. The collapsing suture has a repeating shape having a delivery amplitude in a delivery state of the heart implant device and a deployed amplitude in a deployed state of the heart implant device. A reduced amplitude from the delivery amplitude to the deployed amplitude creates a force causing a portion of the skirt to flip about the first skirt/frame anchor line and to form a sealing portion of the skirt.

The skirt may be anchored to the inflow section of the frame at third skirt/frame anchor points forming a third skirt/frame anchor line. The third skirt/frame anchor line may be longitudinally located between the first and second skirt/frame anchor lines. The skirt may have first and second end sections. The first and second end sections may lie flat in the delivery state. The first end section may be pleated in the deployed state. The second end section may lie flat in the deployed state. The suture/skirt anchor line may be longitudinally located between the outflow section of the frame and the first skirt/frame anchor line in the collapsed state and between the first and second skirt/frame anchor lines in the deployed state. The suture/frame anchor line may be located on or adjacent an inflow end of the frame.

In one embodiment, a heart implant device is disclosed. The heart implant device includes a frame extending along a longitudinal axis and having an inflow section, and an outflow section, and a constricted section extending between the inflow section and the outflow section. The heart implant device further includes a skirt anchored to the inflow section of the frame. The heart implant device also includes extensible sutures anchored to the inflow section of the frame and extending through a sealing portion of the skirt. The extensible sutures are configured to pleat the sealing portion in a deployed state of the frame to form a pleated sealing portion enhancing sealing of the heart implant device and/or reducing paravalvular leak (PVL) in the deployed state.

The extensible sutures may be aligned with the longitudinal axis. The sealing portion defines eyelets for receiving extensible sutures. The extensible sutures may be woven through frame nodes of the frame in a weaving structure. The weaving structure may be an over/under weaving structure.

In another embodiment, a heart implant device is disclosed. The heart implant device includes a frame extending along a longitudinal axis and having an inflow section, and an outflow section, and a constricted section extending between the inflow section and the outflow section. The heart implant device further includes a skirt anchored to the inflow section of the frame. The heart implant device also includes a collapsing suture anchored to the inflow section of the inflow section of the frame at suture/frame anchor points forming a suture/frame anchor line. The collapsing suture is anchored to the skirt at suture/skirt anchor points forming a suture/skirt anchor line coinciding with the suture/frame anchor line. The collapsing suture is configured to collapse to form a sealing portion of the skirt enhancing sealing of the heart implant device and/or reducing leak (PVL) in a deployed state.

The suture/skirt anchor points may include upper suture/skirt anchor points forming an upper suture/skirt anchor line, lower suture/skirt anchor points forming a lower suture/skirt anchor line, and a middle suture/skirt anchor points forming a middle suture/skirt anchor line longitudinally situated between upper suture/skirt anchor line and lower suture/skirt anchor line. The suture/frame anchor line may coincide with the lower suture/skirt anchor line. The suture/frame anchor line may coincide the upper suture/skirt anchor line. The suture/frame anchors may be spaced apart from the suture/skirt anchors.

In yet another embodiment, a heart implant device is disclosed. The heart implant device includes a frame extending along a longitudinal axis and having an inflow section, and an outflow section, and a constricted section extending between the inflow section and the outflow section. The heart implant device further includes a skirt anchored to the inflow section of the frame. The heart implant device includes collapsing sutures including first and second collapsing sutures separate from each other. The first collapsing suture have first and second ends. The second collapsing suture has first and second ends. The first ends of the first and second collapsing sutures are anchored to the skirt. The second ends of the first and second collapsing sutures are anchored to the frame. The first ends have a compressed offset in a delivery state of the frame and a deployed offset in a deployed state of the frame. The deployed offset is greater than the compressed offset causing the skirt to bunch to enhance sealing of the heart implant device and/or reduce paravalvular leak (PVL) in the deployed state.

The second ends of the first and second collapsing sutures may be anchored to the inflow section of the frame. The first and second collapsing sutures may have curved shapes in the compressed and deployed states. The second ends of the first and second collapsing sutures are anchored to the outflow section of the frame. The first and second collapsing sutures have spiral shapes in the compressed and deployed states.

In yet another embodiment, a heart implant device is disclosed. The heart implant device includes a frame extending along a longitudinal axis and having an inflow section, and an outflow section, and a constricted section extending between the inflow section and the outflow section. The heart implant device further includes a skirt anchored to the inflow section of the frame at first skirt/frame anchor points forming a first skirt/frame anchor line and at second skirt/frame anchor points forming a second skirt/frame anchor line. The heart implant device further includes a collapsing suture anchored to the skirt at suture/skirt anchor points forming a suture/skirt anchor line. The collapsing suture is anchored to the inflow section of the frame at suture/frame anchor points forming a suture/frame anchor line. The collapsing suture has a repeating shape having a delivery amplitude in a delivery state of the heart implant device and a deployed amplitude in a deployed state of the heart implant device. The reduced amplitude from the delivery amplitude to the deployed amplitude creates a force causing a portion of the skirt to move closer to the inflow section of the frame in the deployed position.

At least a portion of the skirt may coincide with the constricted section in the delivery state. The skirt may have first and second end sections. The heart implant device may further include a skirt suture anchored to the first and second ends sections of the skirt at first suture/skirt anchor points and second suture/skirt anchor points, respectively. The suture/frame anchor line may coincide with an inflow end of the frame. The portion of the skirt may move closer to the inflow section of the frame in the deployed position is pleated.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a perspective view of a sealing stent graft in a delivery configuration in accordance with one embodiment.

FIG. 2 is a perspective view of the sealing stent graft of FIG. 1 in a deployed configuration in accordance with one embodiment.

FIG. 3 is an enlarged plan view of a region III of the sealing stent graft of FIG. 1 in accordance with one embodiment.

FIG. 4 is an enlarged plan view of a region IV of the sealing stent graft in the deployed configuration of FIG. 2 in accordance with one embodiment.

FIG. 5 is a cross-sectional view of a vessel assembly including a delivery system including the sealing stent graft in the delivery configuration of FIGS. 1 and 3 in accordance with one embodiment.

FIG. 6 is a cross-sectional view of the vessel assembly of FIG. 5 including the sealing stent graft in the deployed configuration of FIGS. 2 and 4 in accordance with one embodiment.

FIG. 7 is a cross-sectional view of a modular assembly including the sealing stent graft in the deployed configuration of FIGS. 2 and 4 in accordance with one embodiment.

FIG. 8 is a cross-sectional view of a sealing stent graft in a delivery configuration in accordance with one embodiment.

FIG. 9 is a cross-sectional view of the sealing stent graft of FIG. 8 in a deployed configuration in accordance with one embodiment.

FIG. 10A depicts a schematic, side view of a transcatheter aortic valve replacement (TAVR) device.

FIG. 10B depicts a schematic, perspective view of the TAVR device shown in FIG. 10A.

FIG. 10C depicts a cross-sectional view of a patient's anatomy and a schematic, side view of a delivery system and the TAVR device of FIGS. 10A and 10B shown in a partially deployed position.

FIG. 10D depicts a cross-sectional view of a patient's anatomy and a schematic, side view of a delivery system and the TAVR device of FIG. 10A and A10B shown in a fully deployed position.

FIG. 11A depicts a cross-sectional view of a portion of a heart implant device (e.g., a TAVR device) in a compressed state (e.g., a delivery state) and having a frame (e.g., an expandable frame of a TAVR device), a skirt anchored to the frame, and a suture anchored to the frame and the skirt.

FIG. 11B depicts a side view of the heart implant device of FIG. 11A in the compressed state.

FIG. 11C depicts a cross-sectional view of a portion of the heart implant device of FIG. 11A in an expanding state (e.g., a partially deployed state).

FIG. 11D depicts a side view of the heart implant device in the expanding state.

FIG. 11E depicts a cross-sectional view of a portion of the heart implant device in an expanded state (e.g., a fully deployed state).

FIG. 11F depicts a side view of the heart implant device in the expanded state.

FIG. 12A depicts a side view of a heart implant device in a compressed state (e.g., a delivery state) and having a frame (e.g., an expandable frame of a TAVR device), a skirt anchored to the frame, and a suture anchored to the frame and the skirt according to another embodiment.

FIG. 12B depicts a side view of the heart implant device of FIG. 12A in an expanding state (e.g., a partially deployed state).

FIG. 12C depicts a side view of the heart implant device of FIG. 12A in an expanded state (e.g., a fully deployed state).

FIG. 13A depicts a side view of heart implant device in a compressed state (e.g., a delivery state) and having a frame (e.g., an expandable frame of a TAVR device), a skirt anchored to the frame, and a suture anchored to the frame and the skirt according to yet another embodiment.

FIG. 13B depicts a side view of the heart implant device of FIG. 13A in an expanding state (e.g., a partially deployed state).

FIG. 13C depicts a side view of the heart implant device of FIG. 13A in an expanded state (e.g., a fully deployed state).

FIG. 14A depicts a schematic, side view of a TAVR device.

FIG. 14B depicts a schematic, side view of a TAVR device having pleated sections and extendable sutures.

FIG. 14C depicts an isolated, side view of the pleated sections and extendable sutures of the TAVR device of 14C.

FIG. 15A depicts a side, schematic view of a frame and a skirt of a heart implant device in a compressed state.

FIG. 15B depicts a side, schematic view of the frame and the skirt of the heart implant device of FIG. 15A in an expanded state.

FIG. 15C is a schematic view depicting a frame node of the frame of a heart implant device (e.g., the heart implant device of FIG. 15A).

FIG. 15D depicts a schematic view of a rivet connecting the frame node to a collapsing suture.

FIG. 15E depicts a schematic view of a suture connecting the frame node to a collapsing suture.

FIG. 15F depicts a schematic view depicting a straight strut of a frame of a heart implant device (e.g., the heart implant device of FIG. 15A).

FIG. 15G depicts a schematic view of a rivet connecting the straight strut to a collapsing suture.

FIG. 15H depicts a schematic view of a suture connecting the straight strut to a collapsing suture.

FIG. 16A depicts a side, schematic view of a frame and a skirt of a heart implant device in a compressed state.

FIG. 16B depicts a side, schematic view of the frame and the skirt of the heart implant device of FIG. 16A in an expanded state.

FIG. 17A depicts a side, schematic view of a frame and a skirt of a heart implant device in a compressed state.

FIG. 17B depicts a side, schematic view of the frame and the skirt of the heart implant device of FIG. 17A in an expanded state.

FIG. 18A depicts a side, schematic view of a frame and a skirt of a heart implant device in a compressed state.

FIG. 18B depicts a side, schematic view of the frame and the skirt of the heart implant device of FIG. 18A in an expanded state.

FIG. 19A depicts a side, schematic view of a heart implant device and a skirt in a compressed state.

FIG. 19B depicts a side, schematic view of the heart implant device and the skirt of FIG. 19A in an expanded state.

FIG. 20A depicts a schematic, side view of an inflow section of a heart implant device in a fully deployed state.

FIG. 20B depicts a schematic, side view of the inflow section of the heart implant device of FIG. 20A in an expanding state (e.g., a partially deployed state).

FIG. 20C depicts a schematic, side view of the inflow section of the heart implant device of FIG. 20A in an expanded state (e.g., a fully deployed state).

FIG. 20D depicts a schematic view of an extensible suture cross weaved axially relative to frame nodes of the frame of the heart implant device shown in FIG. 20A.

FIG. 21A depicts a side view of a heart implant device in a compressed state.

FIG. 21B depicts a side view of an inflow section of the heart implant device of FIG. 21A in an expanding state.

FIG. 21C depicts a side view of the inflow section of the heart implant device in an expanded state.

FIG. 22A depicts a side view of a heart implant device in a compressed state.

FIG. 22B depicts a side view of an inflow section of the heart implant device of FIG. 22A in an expanded state.

FIG. 23A depicts a side view of a heart implant device compressed within a capsule.

FIG. 23B depicts a side view of an inflow section of the heart implant device in FIG. 23A in an expanding state.

FIG. 23C depicts a side view of the heart implant device in an expanded state where the capsule has been released from the heart implant device.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal”is the portion of the stent-graft further from the heart by way of blood flow path.

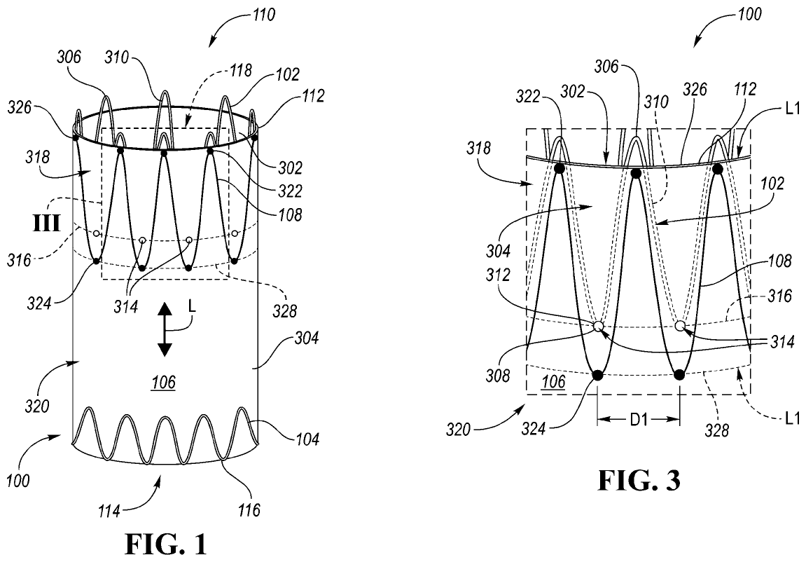

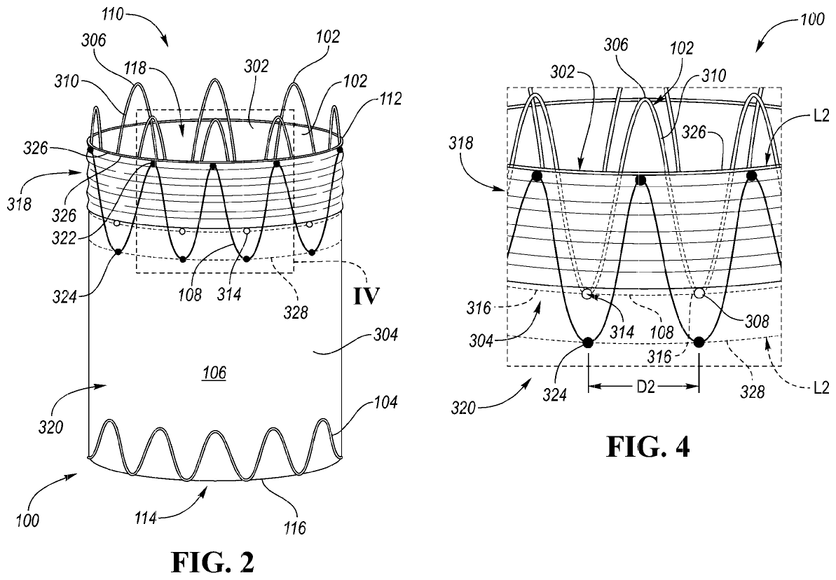

FIG. 1 is a perspective view of a sealing stent graft 100 in a delivery configuration in accordance with one embodiment. FIG. 2 is a perspective view of sealing stent graft 100 of FIG. 1 in a deployed configuration in accordance with one embodiment.

Referring now to FIGS. 1 and 2 together, sealing stent graft 100 includes a proximal stent 102, a distal stent 104, a graft material 106, and a collapsing suture 108. Illustratively, stents 102, 104, sometimes called annular stent rings 102, 104, are self-expanding stents, e.g., nickel titanium alloy (NiTi), sometimes called Nitinol. In another embodiment, stents 102, 104 are balloon expandable. In various embodiments, one or more additional stents are included between stents 102, 104 or only proximal stent 102, e.g., a suprarenal stent, is provided.

In FIG. 1, sealing stent graft 100 is in a delivery configuration (e.g., in a collapsed or constrained configuration), as sealing stent graft 100 is when loaded in a delivery system for delivery to a target location, e.g., to a vessel. In FIG. 2, sealing stent graft 100 is in a deployed configuration (e.g., in an expanded configuration), as sealing stent graft 100 is when deployed at a target location. Expansion of stents 102, 104, e.g., by self-expansion or balloon expansion, causes sealing stent graft 100 to transition from the delivery configuration of FIG. 1 to the deployed configuration of FIG. 2.

Graft material 106 is fabric or a biological tube in accordance with various embodiments. Synthetic materials suitable for use as graft material 106 include DACRON polyester, PTFE, other cloth materials, nylon blends, and polymeric materials. Natural tissue for graft material 106 may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals.

In accordance with this embodiment, graft material 106 includes a proximal opening 110 at a proximal end 112 of graft material 106 and a distal opening 114 at a distal end 116 of graft material 106. Further, sealing stent graft 100 includes a longitudinal axis L. A lumen 118 is defined by graft material 106, and generally by sealing stent graft 100. Lumen 118 extends generally parallel to longitudinal axis L and between proximal opening 110 and distal opening 114 of sealing stent graft 100.

As used herein, the proximal end of a prosthesis such as sealing stent graft 100 is the end closest to the heart via the path of blood flow whereas the distal end is the end furthest away from the heart during deployment. In contrast and of note, the distal end of the catheter is usually identified to the end that is farthest from the operator (handle) while the proximal end of the catheter is the end nearest the operator (handle).

For purposes of clarity of discussion, as used herein, the distal end of the catheter is the end that is farthest from the operator (the end furthest from the handle) while the distal end of the prosthesis is the end nearest the operator (the end nearest the handle), i.e., the distal end of the catheter and the proximal end of the stent graft are the ends furthest from the handle while the proximal end of the catheter and the distal end of the stent graft are the ends nearest the handle. However, those of skill in the art will understand that depending upon the access location, the stent graft and delivery system description may be consistent or opposite in actual usage.

FIG. 3 is an enlarged plan view of a region III of sealing stent graft 100 of FIG. 1 in accordance with one embodiment. Referring now to FIGS. 1 and 3 together, proximal stent 102 is located on an inner surface 302 of graft material 106 whereas collapsing suture 108 is located on an outer surface 304 of graft material 106. Proximal stent 102 includes proximal crowns 306, distal crowns 308, and struts 310 extending between crowns 306, 308 in an alternative repeating arrangement of a sinusoidal ring. Proximal crowns 306 and distal crowns 308 are sometimes called first crowns 306 and second crowns 308.

Proximal stent 102 protrudes proximally past proximal edge 112 of graft material 106. In one or more embodiments, proximal crowns 306 are proximal of proximal edge 112 and distal crowns 308 are distal of proximal edge 112 such that proximal stent 102 extends from distal crowns 308 proximally past proximal edge 112 of graft material 106 to proximal crowns 306. Accordingly, proximal edge 112 of graft material 106 overlaps struts 310. In another embodiment, proximal edge 112 of graft material 106 extend proximally past the entire proximal stent 102. For example, in the delivery configuration, graft material 106 may overlap at least 25%, 33%, 50 %, 67%, or 75, generally 20% to 150%, of the height of proximal stent 102 (e.g., from distal crown 308 to proximal crown 306).

Distal crowns 308 are attached to graft material 106, e.g., by sutures 312, at anchor points 314. In other words, at each specific location where a distal crown 308 is sutured to graft material 106 by a suture 312, an anchor point 314 is defined. Accordingly, anchor points 314 are located circumferential around the entire circumference of graft material 106 at a particular longitudinal distance from proximal edge 112 of graft material 106. An anchor line 316 is defined as a circle that extends through and connects anchor points 314.

Graft material 106 is divided into a sealing skirt 318 and an exclusion section 320 by anchor line 316. More particularly, sealing skirt 318 is defined as the section of graft material 106 proximal of anchor line 316 and exclusion section 320 is defined as the section of graft material 106 distal of anchor line 316. Sealing skirt 318 and exclusion section 320 meet at anchor line 316. Sealing skirt 318 is excess or loose graft material 106 that extends between anchor line 316 and proximal edge 112.

Collapsing suture 108, e.g., a suture, is attached to graft material 106 at proximal suture points 322 and distal suture points 324, sometimes called first periodic points 322 and second periodic points 324, respectively, in an alternating repeating pattern. For example, collapsing suture 108 is passed from outer surface 304 to inner surface 302 through graft material 106 and then back from inner surface 302 to outer surface 304 at each suture point 322, 324. While referred to as a suture herein, collapsing suture 108 may be any filament or thread-like material, such as a polymer, metal, or natural material, and is not limited to surgical sutures.

Proximal suture points 322 are located on sealing skirt 318 on or adjacent proximal end 112 of graft material 106. Proximal suture points 322 are located circumferential around the entire circumference of graft material 106. A first or proximal suture line 326 is defined as a circle that extends through and connects proximal suture points 322. Proximal suture points 322 overlap proximal stent 102 in this embodiment.

In this embodiment where proximal suture points 322 are located on proximal end 112 of graft material 106, proximal suture line 326 lies upon proximal end 112. However, in another embodiment, proximal suture points 322 are directly adjacent, but not directly on, proximal end 112 and so a small longitudinal space exists between proximal suture line 326 and proximal end 112.

Distal suture points 324 are located on graft material 106 on or adjacent anchor line 316. Distal suture points 324 are located circumferential around the entire circumference of graft material 106. A second or distal suture line 328 is defined as a circle that extends through and connects distal suture points 324.

In this embodiment, distal suture line 328 lies upon exclusion section 320 directly adjacent and distal of anchor line 316. However, in other embodiments, distal suture points 324, and thus distal suture line 328, lie directly on or are directly adjacent and proximal of anchor line 316.

Between each adjacent proximal suture point 322 or distal suture point 324, a circumferential distance D1 exists. Circumferential distance D1 is a distance along the circumference of graft material 106. Further, a longitudinal distance L1 exists between proximal suture points 322 and distal suture points 324. Longitudinal distance L1 is a distance in the longitudinal direction, i.e., in a direction parallel with longitudinal axis L of graft material 106. Distances D1, L1 as well as the number of suture points 322, 324 define a fixed length of collapsing suture 108, which is a long suture loop in this embodiment.

Further, to selectively control the amount of collapse of sealing skirt 318, the number of suture points 322, 324 and the positioning of suture points 322, 324 can be tailored. For example, FIGS. 3 and 4 illustrate a one-to-one relationship between the number of distal suture points 324 and anchor points 314, i.e., one distal suture point 324 for every one anchor point 314. However in other embodiments, more or less than a one-to-one relationship between the number of distal suture points 324 and anchor points 314 is used, e.g., to create more/less collapse of sealing skirt 318. For example, the ratio of distal suture points 324 to anchor points 314 may be 2:1 or 3:1, or vice versa.

Further, FIGS. 3 and 4 illustrate that distal suture points 324 are aligned with anchor points 314, i.e., are directly below anchor points 314 and have the same clocking or radial orientation. However, in other embodiments, distal suture points 324 have other radial orientations with respect to anchor points 314, e.g., are located between anchor points 314. In yet another embodiment, distal suture points 324 have no particular radial orientation with respect to anchor points 314.

Sealing skirt 318 is excess or loose graft material 106 that extends proximal of anchor line 316. As illustrated in FIGS. 1 and 3, in the delivery configuration, sealing skirt 318 lays flat upon proximal stent 102 as a cylinder, i.e., tube, in a state the same or identical as a state of exclusion section 320. Accordingly, sealing skirt 316 does not impact the packing density of sealing stent graft 100 when proximal stent 102 is collapsed or compresses thus maximizing the range of anatomical applications as well as reducing the complexity of the procedure. The addition of collapsing suture 108 adds negligibly or at most minimally to the packing density.

FIG. 4 is an enlarged plan view of a region IV of sealing stent graft 100 in the deployed configuration of FIG. 2 in accordance with one embodiment. During deployment, proximal stent 102 expanding causes sealing stent graft 100 to transition from the delivery configuration of FIGS. 1, 3 to the deployed configuration of FIGS. 2, 4 and collapses sealing skirt 318 to be ruffled, folded, or pleated. More particularly, expansion of proximal stent 102 causes collapsing suture 108 to pull sealing skirt 318 down towards distal crowns 308 causing sealing skirt 318 to bulge outwards, increasing the diameter of sealing skirt 318 relative to exclusion section 320.

In more detail, as proximal stent 102 expands and the circumference of graft material 106 increases, circumferential distance D1 between adjacent proximal suture points 322 and distal suture points 324 increases to a greater circumferential distance D2. As collapsing suture 108 has a fixed length, the increase in circumferential distance D1 to circumferential distance D2 causes a relative decrease in longitudinal distance L1 to a longitudinal distance L2 between proximal suture points 322 and distal suture points 324, longitudinal distance L2 being less than longitudinal distance L1. Generally, expansion of proximal stent 102 causes longitudinal distance L1 between proximal suture line 326 and distal suture line 328 to decrease to longitudinal distance L2.

Distal suture points 324 are fixed in location on graft material 106 due to the proximity to anchor points 314. More particularly, graft material 106 is attached to distal crowns 308 at anchor points 314 and thus cannot move relative to proximal stent 102 along anchor line 316. As distal suture points 324 are on or adjacent anchor line 316, distal suture points 324 also cannot move relative to proximal stent 102.

In contrast, proximal suture points 322 are free to move distally along with the attached sealing skirt 318 relative to proximal stent. 102 and anchor line 316. Accordingly, as the longitudinal distance between proximal suture points 322 and distal suture points 324 decreases from longitudinal distance L1 (FIG. 3) to longitudinal distance L2 (FIG. 4), proximal suture points 322 and the attached sealing skirt 318 move distally. This longitudinally collapses and compresses sealing skirt 318 on proximal stent 102 between proximal suture points 322 and anchor line 316. For example, when longitudinally collapsed and compressed, the length of sealing skirt 318 may be reduced by 10% to 90% and sealing skirt may overlap 5% to 90% of the height of proximal stent 102 (e.g., from distal crown 308 to proximal crown 306).

The collapse of sealing skirt 318 causes sealing skirt 318 to become ruffled/folded/pleated and to protrude radially outward. For example, sealing skirt 318 has longitudinal corrugations, e.g., a series of peaks and troughs, that protrude outward from proximal stent 102. This thickening of sealing skirt 318 improves sealing of sealing skirt 318, and more generally of sealing stent graft 100, with the vessel or other prosthesis in which sealing stent graft 100 is deployed. For example, the excess fabric of sealing skirt 318 can help achieve seals at short seal length and non-circular aortic vessels (e.g., reduce type 1a endoleaks). This enhanced sealing is achieved without requiring new materials, new stent designs, new manufacturing processes, or different catheter systems.

As set forth above, collapsing suture 108, graft material 106, and proximal stent 102 are utilized to provide a passive mechanism that maintains a low profile transcatheter system for delivery and improves the sealing feature on sealing stent graft 100, sometimes called the implant, during deployment. This reduces procedural complexity and as well as improves in-vivo performance. Sealing skirt 318 improves sealing performance in normal anatomies and in complex anatomies such as non-circular, calcified, and short landing zone anatomies.

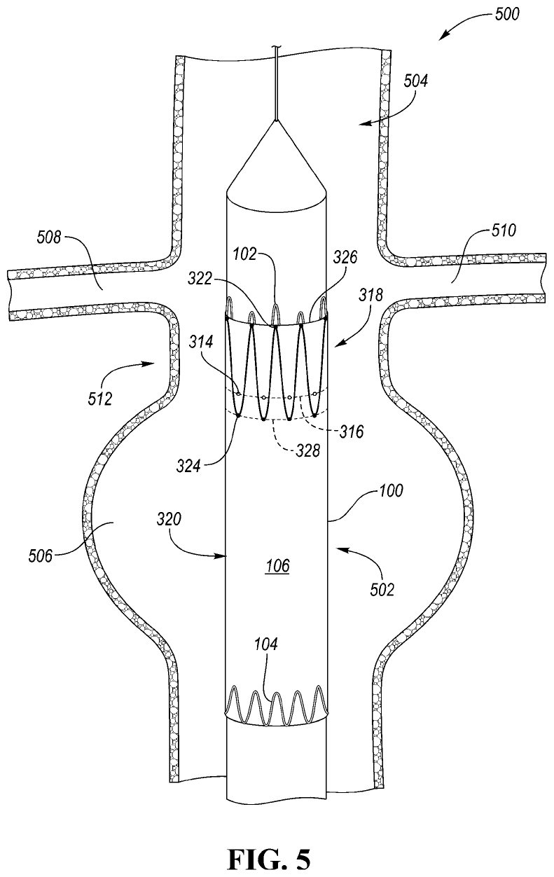

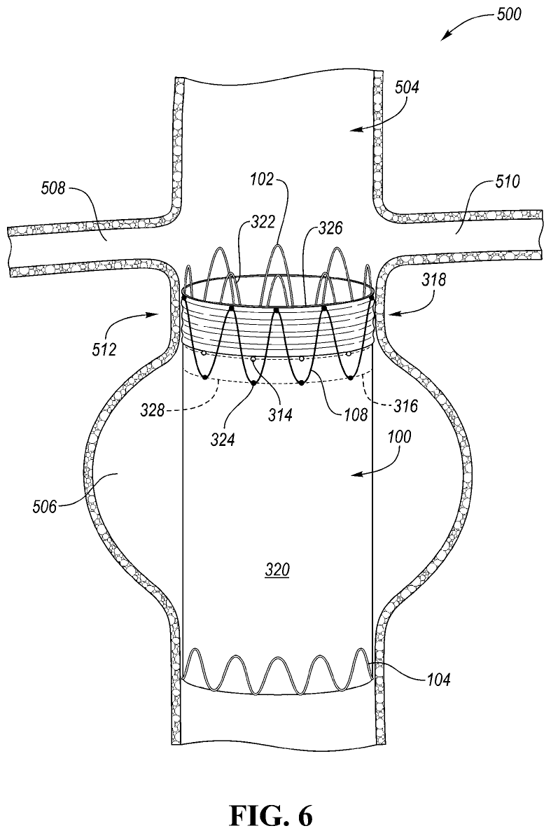

FIG. 5 is a cross-sectional view of a vessel assembly 500 including a delivery system 502 including sealing stent graft 100 in the delivery configuration of FIGS. 1 and 3 in accordance with one embodiment. FIG. 6 is a cross-sectional view of vessel assembly 500 of FIG. 5 including sealing stent graft 100 in the deployed configuration of FIGS. 2 and 4 in accordance with one embodiment. Sealing stent graft 100 is positioned within a vessel 504 using delivery system 502 (FIG. 5) and deployed into vessel 504 to exclude an aneurysm 506 (FIG. 6) using any one of a number of techniques well known to those of skill in the art.

More particularly, vessel 504, e.g., the aorta, includes aneurysm 506. Emanating from vessel 504 is a first branch vessel 508 and a second branch vessel 510, sometimes called visceral branches of the abdominal aorta. The location of branch vessels 508, 510 vary from patient to patient. Examples of branch vessels include the renal arteries (RA) and the superior mesenteric artery (SMA).

Sealing stent graft 100 is delivered in the delivery configuration of FIGS. 1 and 3 to the deployment location in vessel 504 within delivery system 502 as shown in FIG. 5. Sealing stent graft 100 is deployed from delivery system 502 just distal of branch vessels 508, 510 and delivery system 502 is removed as shown in FIG. 6.

Sealing skirt 318 is deployed in a landing zone 512 between branch vessels 508, 510 and aneurysm 506. In one embodiment, landing zone 512 is short, thus providing minimal area for sealing. However, due to the outward bulging and ruffles of sealing skirt 318, sealing skirt 318 forms a good seal with short landing zone 512.

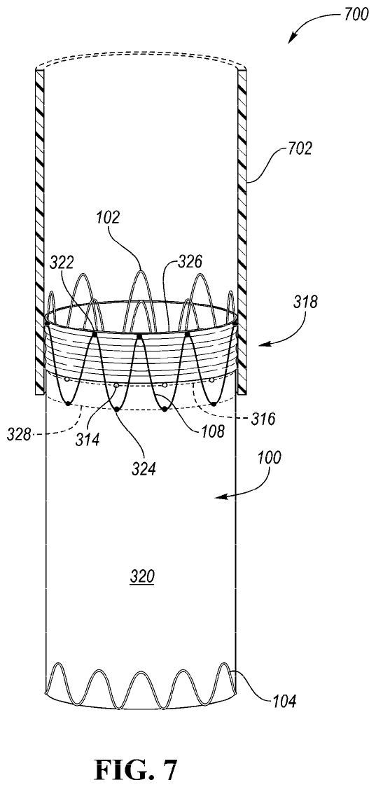

Although FIGS. 5 and 6 illustrate formation of a seal between sealing skirt 318 and vessel 504, in another embodiment, sealing skirt 318 is used to form an enhanced seal with other vessels or structures. Illustratively, sealing stent graft 100 is utilized with transcatheter cardiovascular therapies that require sealing, e.g., endovascular repair, heart valve repair, and so forth. In another embodiment, sealing stent graft 100 is used with another prosthesis such as a stent graft to form a modular assembly. Such an example is discussed below with reference to FIG. 7.

FIG. 7 is a cross-sectional view of a modular assembly 700 including sealing stent graft 100 in the deployed configuration of FIGS. 2 and 4 in accordance with one embodiment. Referring to FIG. 7, modular assembly 700 includes a first component 702 and sealing stent graft 100 deployed within first component 702. Illustratively, first component 702, e.g., a stent graft, is deployed initially. Sealing stent graft 100 is then located within first component 702 and then deployed. Sealing skirt 318 bulges outwards and into first component 702 thus providing a robust seal between first component 702 and sealing stent graft 100.

Although sealing stent graft 100 is discussed and illustrated as having sealing skirt 318 at the proximal end of sealing stent graft 100, in another embodiment, sealing skirt 318 is provided at the distal end of sealing stent graft 100. In accordance with this embodiment, the terms “proximal” and “'distal” as used to describe sealing stent graft 100 above are replaced with the terms “distal” and “proximal”, respectively. In this embodiment, a distal end of the stent graft is pulled proximally by the collapsing suture to form the sealing skirt 318. Forming a distal sealing skirt may improve a seal with the vessel wall at the distal end of the stent graft (e.g., reduce type 1b endoleaks) or it may improve a seal with a proximal end of another stent graft component (similar to FIG. 7 but wherein sealing stent graft 100 is proximal to first component 702). In another embodiment, the sealing stent graft 100 may have a sealing skirt 318 at both the proximal and distal ends.

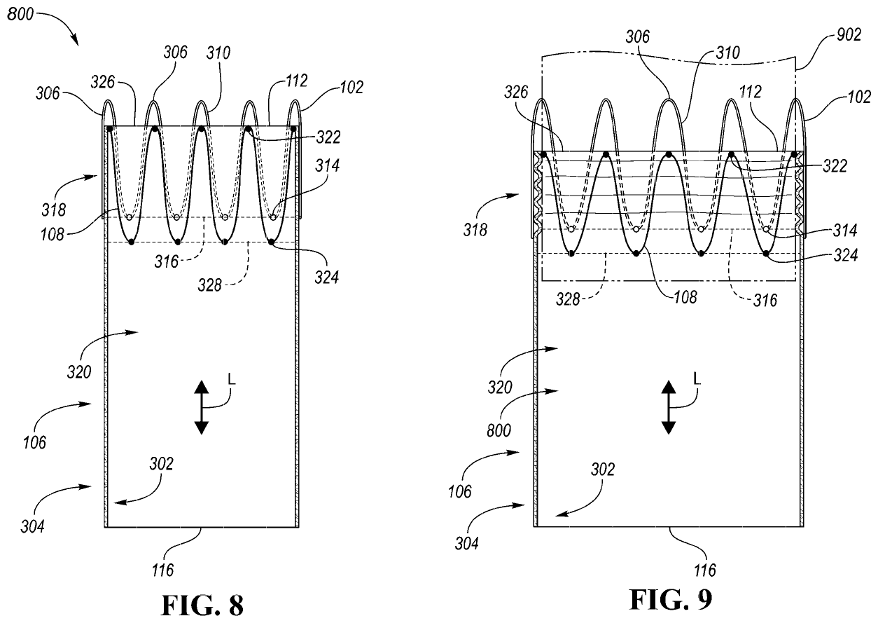

Further, although sealing skirt 318 is set forth as bulging outward upon collapse in the above embodiments, in another embodiment, sealing skirt 318 bulges inward upon collapse. Such an example is set forth in FIGS. 8 and 9.

FIG. 8 is a cross-sectional view of a sealing stent graft 800 in a delivery configuration in accordance with one embodiment. FIG. 9 is a cross-sectional view of sealing stent graft 800 of FIG. 8 in a deployed configuration in accordance with one embodiment. In the view of FIGS. 8 and 9, stent graft 800 has been cut in half longitudinally to allow visualization of collapsing suture 108 located on inner surface 302 of graft material 106. Stent graft 800 of FIGS. 8 and 9 is similar to stent graft 100 of FIGS. 1-4 and only the significant differences are discussed below.

In accordance with this embodiment, proximal stent 102 is located on outer surface 304 of graft material 106 whereas collapsing suture 108 is located on inner surface 302 of graft material 106. Accordingly, during deployment, sealing skirt 318 is pulled downward by collapsing suture 108 on the inside of proximal stent 102 and thus bulges inward as illustrated in FIG. 9.

Inward bulging sealing skirt 318 provides enhanced sealing with structures located inside of sealing stent graft 800. In one embodiment, a modular assembly includes a first component 902 as indicated in the phantom line coupled inside of sealing stent graft 800. For example, sealing stent graft 800 is deployed initially such that sealing skirt 318 bulges inwards. First component 902 is then then located within sealing stent graft 800 to protrude proximally from sealing stent graft 800 and then deployed. Sealing skirt 318 bulges inward and into first component 902 thus providing a robust seal between sealing stent graft 800 and first component 902. Similar to the outward bulging sealing skirt 318, the inward bulging sealing skirt may be located at the proximal and/or distal end of the sealing stent graft 800.

While embodiments herein have been described with respect to a stent graft, e.g., for use in treating aortic aneurysms, the passive sealing skirt may be applied to any expanding catheter-based device, such as transcatheter heart valve replacement or repair devices. For example, the sealing skirt may be used with a transcatheter aortic valve replacement (TAVR) device, such as a self-expanding TAVR device. Such devices generally include a frame (e.g., a stent frame) and a prosthetic valve and may optionally include a skirt material to reduce paravalvular leak (PVL). These skirts are typically sewn to the frame in a static manner, however, the passive sealing skirt disclosed herein may be incorporated into such a device. The passive sealing skirt may provide the same advantages described herein to a TAVR device or other heart valve device, for example, improved sealing and reduced profile in the delivery configuration. For a TAVR device, the skirt may be positioned at the inflow or proximal end of the device. As used herein, the term graft material may also apply to sealing skirt materials for heart valve replacement or repair devices. The passive sealing skirt may replace existing skirt(s) on transcatheter heart valve replacement or repair devices or may be added theretofore improved sealing.

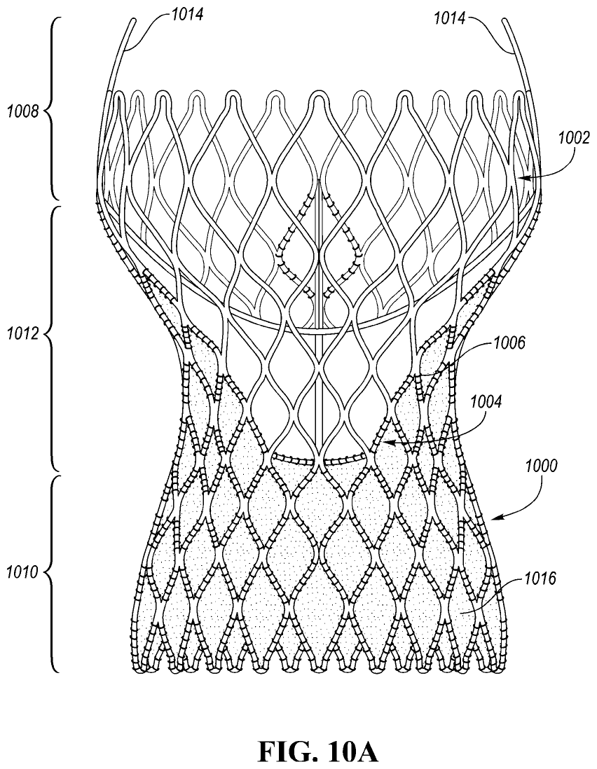

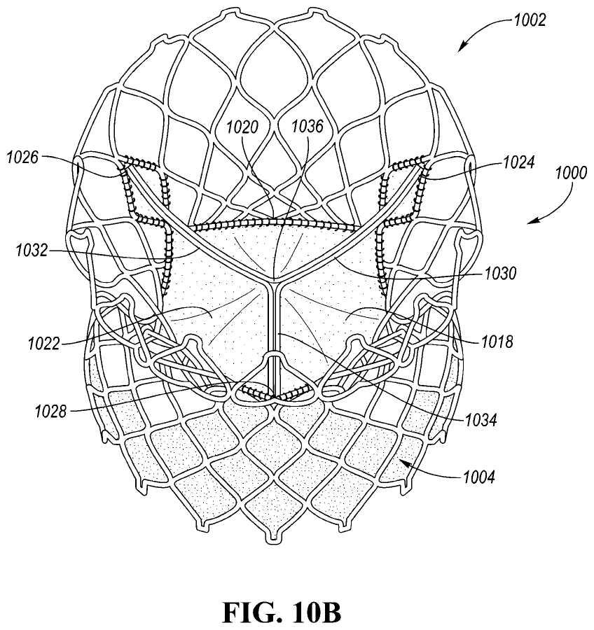

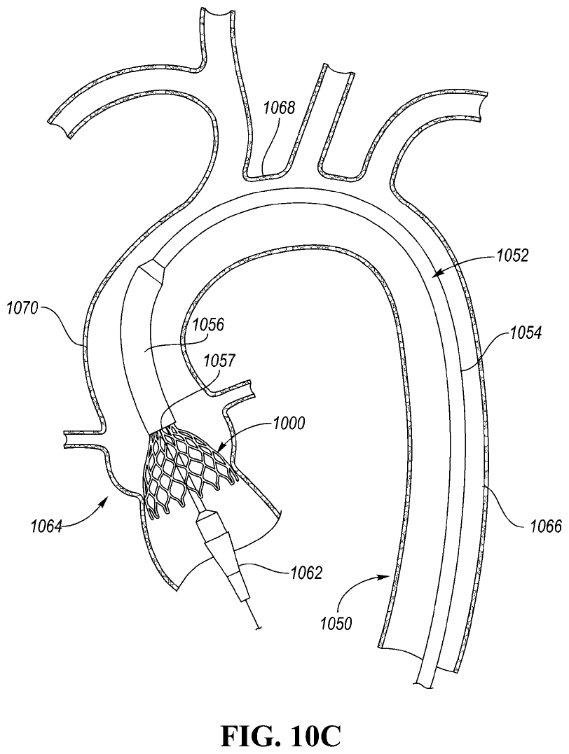

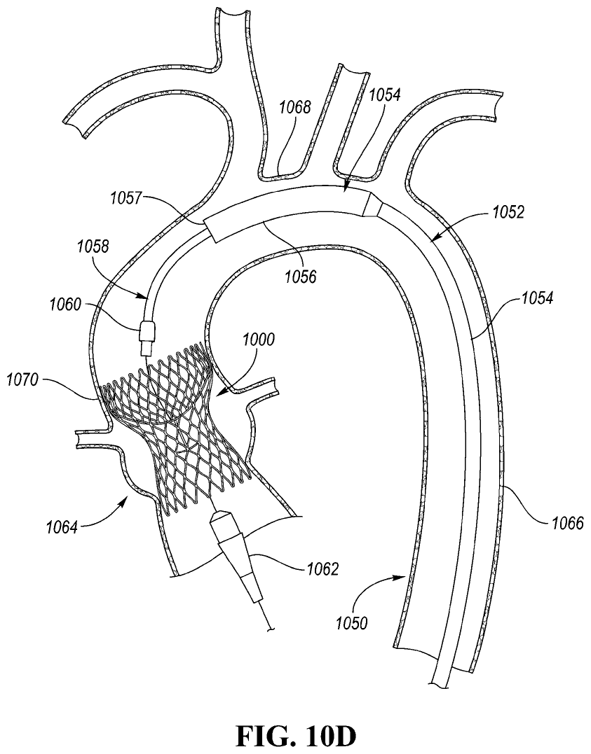

FIG. 10A depicts a schematic, side view of transcatheter aortic valve replacement (TAVR) device 1000. FIG. 10B depicts a schematic, perspective view of TAVR device 1000. As shown in FIGS. 10A and 10B, TAVR device 1000 includes expandable frame 1002 and valve body 1004. Valve body 1004 may be secured to an interior surface of expandable frame 1002 via sutures 1006 or other type of fastener. Valve body 1004 may be formed from a natural or synthetic material. Non-limiting examples of such materials include mammalian tissue (e.g., porcine, equine, or bovine pericardium, synthetic material, or polymeric material). Expandable frame 1002 may be a self-expanding structure formed by laser cutting or etching a tube formed of a metal alloy (e.g., stainless steel alloy, nickel titanium alloy, Nitinol, or other shape memory material). Alternatively, the expandable frame 1002 may be a balloon-expandable frame. As shown in FIGS. 10A and 10B, TAVR device 1000 is in an expanded, deployed state (e.g., when TAVR device 1000 is deployed at a target location).

Expandable frame 1002 may include outflow section 1008, inflow section 1010, and constriction region 1012 extending between outflow section 1008 and inflow section 1010. Expandable frame 1002 may include cells having sizes that may vary along the length of the prosthesis. Upon deployment of TAVR device 1000 within the vasculature of a patient, inflow section 1010 may extend into and anchor within the aortic annulus of a patient's left ventricle and outflow section 1008 may be positioned in the patient's ascending aorta. Expandable frame 1002 may also include paddles or eyelets 1014 configured for loading TAVR device 1000 into a delivery system as described herein.

Valve body 1004 may include skirt 1016. As shown in FIGS. 10A and 10B, skirt 1016 is sewn to expandable frame 1002 in a static manner. Valve body 1004 may further include first leaflet 1018, second leaflet 1020, and third leaflet 1022, which may be attached along their bases to skirt 1016 with fasteners (e.g., sutures or a biocompatible adhesive). First leaflet 1018, second leaflet 1020, and third leaflet 1022 form three adjoining pairs of leaflets (i.e., first and second leaflets 1018 and 1020; second and third leaflets 1020 and 1022; and third and first leaflets 1022 and 1018). Each of the adjoining pairs of leaflets may be attached to each other at their lateral ends to form first commissure 1024, second commissure 1026, and third commissure 1028, respectively. First free edges 1030 between first and second leaflets 1018 and 1020; second free edges 1032 between second and third leaflets 1020 and 1022; and third free edges 1034 between third and first leaflets 1022 and 1018, form coaptation edges that meet in coaptation area 1036.

FIG. 10C depicts a cross-sectional view of patient's anatomy 1050 and a schematic, side view of delivery system 1052 and TAVR device 1000 in a partially deployed position. FIG. 10D depicts a cross-sectional view of patient's anatomy 1050 and a schematic, side view of delivery system 1052 and TAVR device 1000 in a fully deployed position. Delivery system 1052 includes delivery sheath assembly 1054, capsule 1056, inner shaft assembly 1058, retention hub 1060, and tip 1062.

Delivery system 1052 is configured to retain TAVR device 1000 in a delivery state in which TAVR device 1000 is loaded in a constrained position within delivery system 1052. In the delivery state, TAVR device 1000 is coupled to inner shaft assembly 1058 via retention hub 1060 (e.g., eyelets 1014 of TAVR device 1000 are captured by retention hub 1060) and is compressively retained within capsule 1056 of delivery sheath assembly 1054. Loaded delivery system 1052 may be configured to percutaneously deliver TAVR device 1000 to an implantation site (e.g., a defective heart valve). As shown in FIGS. 10C and 10D, loaded delivery system 1052 may be advanced toward implantation site 1064 in a retrograde manner through the patient's femoral artery into descending aorta 1066 over aortic arch 1068 through ascending aorta 1070 and across a defective heart valve (e.g., about mid-way through defective heart valve) at implantation site 1064.

For self-expanding embodiments, delivery sheath assembly 1054 is configured to withdraw capsule 1056 proximally from TAVR device 1000 via operation of a handle (not shown) on delivery system 1052, thereby allowing TAVR device 1000 to expand from the constrained position to a partially deployed position shown in FIG. 10C and fully deployed position shown in FIG. 10D in which TAVR device 1000 is fully released from capsule 1056 and TAVR device 1000 is implanted to the native valve. A release sheath assembly of delivery system 1052 may be configured to fully release TAVR device 1000 from capsule 1056. The handle may be configured to maneuver capsule 1056 to the partially deployed position shown in FIG. 10C in which a distal region of TAVR device 1000 is permitted to self-expand at distal end 1057 of retention hub 1056 while a proximal region of TAVR device 1000 remains coupled to retention hub 1056.

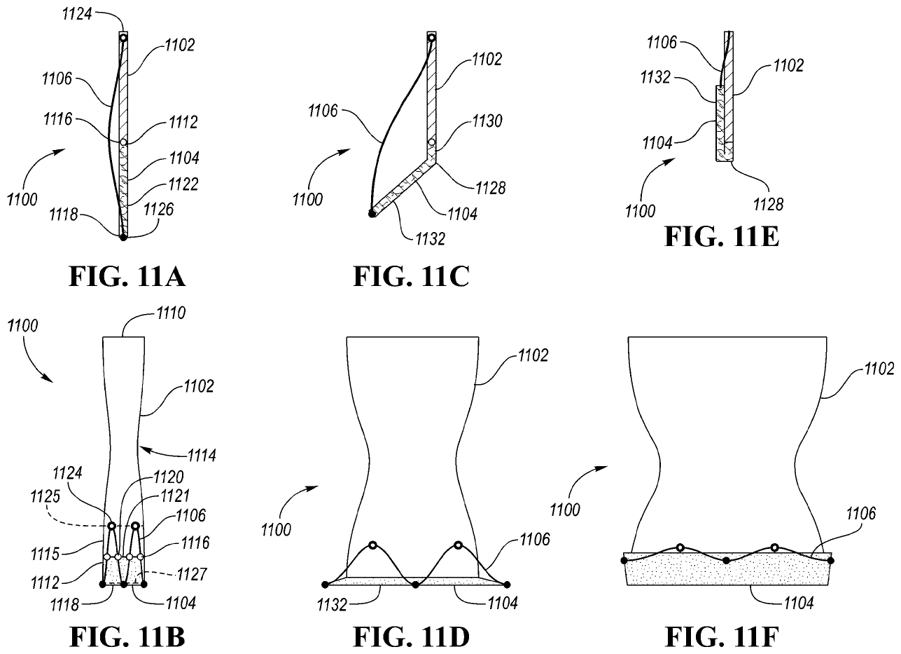

FIG. 11A depicts a cross-sectional view of a portion of heart implant device 1100 (e.g., a TAVR device) in a compressed state (e.g., a delivery state). FIG. 11B depicts a side view of heart implant device 1100 in the compressed state (e.g., the delivery state). Heart implant device 1100 includes frame 1102 (e.g., an expandable frame of a TAVR device), skirt 1104 anchored to frame 1102, and collapsing suture 1106 anchored to frame 1102 and skirt 1104. Skirt 1104 may replace skirt 1016 shown in FIG. 10A or may be an additional skirt on top of (e.g., outward) skirt 1016.

Frame 1102 includes outflow end 1110, inflow end 1112 and frame body 1114 extending therebetween. Skirt 1104 includes first end 1116, second end 1118, and skirt body 1122 extending therebetween. Skirt body 1122 may be formed of a cylindrical body (e.g., tube). Collapsing suture 1106 may be formed any filament or thread-like material, such as a polymer, metal, or natural material, and is not limited to surgical sutures.

Inflow end 1112 of frame 1102 is secured to first end 1116 of skirt 1104 via anchor points 1120 to form seam 1121 circumferentially around a circumference (e.g., the entire circumference) of heart implant device 1100. Anchor points 1120 may include sutures securing frame 1102 and skirt 1104 together. Seam 1121 may form a circle that extends through anchor points 1120. Anchor points 1120 may be spaced evenly apart from each other around a circumference (e.g., the entire circumference) of frame 1102 to reinforce the connection between frame 1102 and skirt 1104. As shown in FIGS. 11A and 11B, skirt body 1122 extends away from inflow end 1112 and is aligned with inflow section 1115 of frame body 1114 such that skirt 1104 does not impact the packing density of heart implant device 1100 in the compressed state with collapsing suture 1106 adding negligibly or at most minimally to the packing density.

Collapsing suture 1106 is secured to frame body 1114 at anchor points 1124 and is secured to second end 1118 of skirt 1104 at anchor points 1126 in a periodic, alternating repeating pattern. In one or more embodiments, anchor points 1124 and anchor points 1126 secure collapsing suture 1106 to outside surfaces of frame body 1114 and skirt 1104, respectively, such that collapsing suture 1106 is located outward these outside surfaces. Anchor points 1124 may be located circumferentially around a circumference (e.g., the entire circumference) of frame body 1114 to form circle 1125 including anchor points 1124. Circle 1125 is spaced apart from inflow end 1112 of frame 1102 toward outflow end 1110 of frame 1102, and the spaced apart distance may be greater than the length of skirt 1104. Anchor points 1126 may be located circumferentially around a circumference (e.g., the entire circumference) of second end 1118 of skirt 1104 to form second circle 1127 including anchor points 1126.

As shown in FIG. 11B, collapsing suture 1106 forms a sinusoidal ring having a delivery amplitude and a delivery period in the delivery state. In other embodiments, collapsing suture 1106 may have a zig-zag shape of alternating peaks and valleys with legs therebetween. The delivery amplitude may refer to a distance between seam 1121 and a peak at an anchor point 1124 or a valley at an anchor point 1126 of collapsing suture 1106. The delivery period may refer to a distance between adjacent peaks (e.g., adjacent peaks at adjacent anchor points 1124) or adjacent valleys (e.g., adjacent valleys at adjacent anchor points 1126) of collapsing suture 1106. In the delivery state, heart implant device 1100 has a compressed nominal diameter and a compressed length.

FIG. 11C depicts a cross-sectional view of a portion of heart implant device 1100 in an expanding state (e.g., a partially deployed state). FIG. 11D depicts a side view of heart implant device 1100 in the expanding state. As shown in FIG. 11D, frame 1102 has partially deployed from the delivery state into a partially deployed nominal diameter and a partially deployed length. The partially deployed nominal diameter is greater than the compressed nominal diameter. The partially deployed length is less than the compressed length (e.g., due to foreshortening from radial expansion). The increased nominal diameter and the decreased length of frame 1102 between the compressed state and the partially deployed state reduces the amplitude of the sinusoidal ring of collapsing suture 1106 to a partially deployed amplitude and increases the period of the sinusoidal ring of collapsing suture 1106 to a partially deployed period. These changes in the amplitude and the period of the sinusoidal ring of collapsing suture 1106 cause collapsing suture 1106 to bulge outward because collapsing suture 1106 is connected at its ends to anchor points 1124 and 1126. This bulging outward movement causes skirt 1104 to rotate at hinge line 1128. As shown in FIG. 11C, hinge line 1128 is spaced apart from seam 1121 because anchor points 1120 reinforce seam 1121 so that it hinges at a point spaced apart from seam 1121. In other embodiments, hinge line 1128 may coincide with anchor point 1120 or hinge line 1128 may be further spaced from seam 1121.

Hinge line 1128 separates skirt 1104 into first section 1130 extending between seam 1121 and hinge line 1128 and second section 1132 extending between hinge line 1128 and second end 1118 of skirt 1104. In this partially deployed state, first section 1130 is aligned with inflow section 1115 of frame body 1114 and second section 1132 bulges or rotates outward first section 1130 and frame body 1114 about hinge line 1128.

FIG. 11E depicts a cross-sectional view of a portion of heart implant device 1100 in an expanded state (e.g., a fully deployed state). FIG. 11F depicts a side view of heart implant device 1100 in the expanded state. As shown in FIG. 11F, frame 1102 has fully deployed from the partially deployed state into a fully deployed nominal diameter and a fully deployed length. The fully deployed nominal diameter is greater than the other nominal diameters. The fully deployed length is less than the other lengths. The increase in nominal diameter and decrease in length of frame 1102 further decreases the amplitude of the sinusoidal ring and increases the period of the sinusoidal ring. These changes in the amplitude and the period of the sinusoidal ring of collapsing suture 1106 causes collapsing suture 1106 to draw inward from its position in the partially deployed state thereby further rotating skirt 1104 at hinge line 1128 such that a surface of second section 1132 contacts the outer surface of frame 1102. In the fully deployed state, second section 1132 and a portion of frame 1102 overlap to form a region of increased radial thickness configured to enhance sealing of heart implant device 1100 and/or to reduce paravalvular leak (PVL). There may also be a region where second section 1132 and first section 1130 of the skirt contacts each other to form a doubled-up section of the skirt with increased (e.g., 2×) thickness.

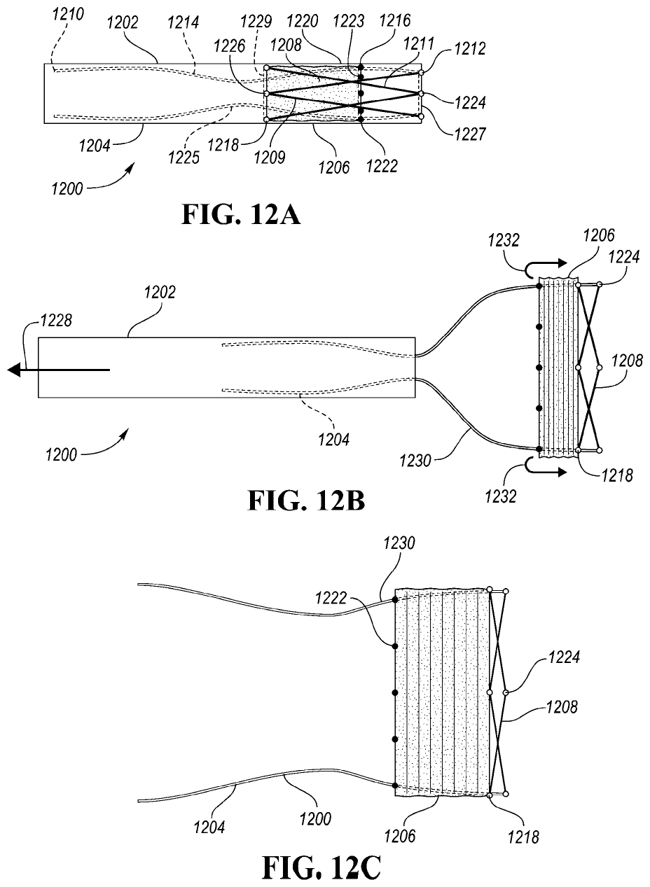

FIG. 12A depicts a side view of heart implant device 1200 in a compressed state (e.g., a delivery state). As shown in FIG. 12A, heart implant device 1200 is compressed within capsule 1202. Heart implant device 1200 includes frame 1204 (e.g., an expandable frame of a TAVR device), skirt 1206 anchored to frame 1204, and collapsing suture 1208 anchored to frame 1204 and skirt 1206. Skirt 1204 may replace skirt 1016 shown in FIG. 10A or may be an additional skirt on top of (e.g., outward) skirt 1016.

Frame 1204 includes outflow end 1210, inflow end 1212, and frame body 1214 extending therebetween. Skirt 1206 includes first end 1216, second end 1218, and skirt body 1220 extending therebetween. Skirt body 1220 may be formed of a cylindrical body (e.g., a tube). Collapsing suture 1208 may be formed of any filament or thread-like material, such as a polymer, metal, or natural material, and is not limited to surgical sutures.

First end 1216 of skirt 1206 is secured to frame body 1214 of frame 1204 via anchor points 1222 in a repeating pattern. Anchor points 1222 may include sutures securing skirt 1206 and frame 1204 together. Anchor points 1222 may be located circumferentially around a circumference (e.g., the entire circumference) of frame body 1214 to form circle 1223 at first end 1216 of skirt 1206 including anchor points 1222. Anchor points 1222 may be spaced evenly apart from each other around a circumference (e.g., the entire circumference) of frame body 1214 to reinforce the connection between frame 1204 and skirt 1206. As shown in FIG. 12A, skirt body 1220 extends away from first circle 1223 toward constricted portion 1225 of frame 1204 thereby occupying a reduced diameter portion of frame body 1214 such that skirt 1206 does not impact or only negligibly impacts the packing density of heart implant device 1200 in the compressed state within capsule 1202.

Collapsing suture 1208 is secured to inflow end 1212 of frame 1204 at anchor points 1224 and is secured to second end 1218 of skirt 1206 at anchor points 1226 in a periodic, alternating repeating pattern. As shown in FIG. 12A, anchor points 1224 and anchor points 1226 secure collapsing suture 1208 to an outside surface of frame 1204 and an outside surface of skirt 1206, respectively, such that collapsing suture 1206 is located outward skirt 1206. In another embodiment, anchor points 1224 and anchor points 1226 secure collapsing suture 1208 to an outside surface of frame 1204 and an inside surface of skirt 1206, respectively, such that collapsing suture 1206 is located between frame 1204 and skirt 1206.

Anchor points 1224 may be located circumferentially around a circumference (e.g., the entire circumference) of inflow end 1212 of frame 1204 to form circle 1227 including anchor points 1224. Anchor points 1226 may be located circumferentially around a circumference (e.g., the entire circumference) of second end 1218 of skirt 1206 to form circle 1229 including anchor points 1226.

In one or more embodiments, the collapsing suture comprises a first suture having a repeating shape and a second suture having a repeating shape. FIG. 12A schematically illustrates collapsing suture 1208 having first sinusoidal suture 1209 and second sinusoidal suture 1211 (but in other embodiments the first and second sutures may be zig-zag shaped rings that form ring shaped scissor arms). Alternating peaks and valleys of first sinusoidal suture 1209 are secured via alternating anchor points 1224 and 1226 and alternating peaks and valleys of second sinusoidal suture 1211 are secured via alternating anchor points 1226 and 1224 such that first and second sinusoidal sutures 1209 and 1211 are out of phase with each other (e.g., the peak of one aligns with the valley of the other). While FIG. 12A shows first and second sinusoidal sutures 1209 and 1211 out of phase 180 degrees, the two or more sutures may be out of phase a different number of degrees, e.g., 60 degrees or 120 degrees.

The use of two or more sutures provides a longer axial travel than a single suture having the same number of total anchor points, thereby allowing for a greater distance to be created from inflow end 1212 to attachment of second end 1218 of skirt 1206 during loading (e.g., the delivery sate). The use of two or more sutures may also increase the strength when pulling skirt 1206 toward inflow end 1212.

First and second sinusoidal sutures 1209 and 1211 may have a delivery amplitude and a delivery period in the delivery state (in other embodiments, first and second sinusoidal sutures 1209 and 1211 may have different amplitudes and periods). The delivery amplitude may refer to a distance between inflow end 1212 of frame 1204 at first anchor points 1224 and second end 1218 of skirt 1206 at second anchor points 1226. The delivery period may refer to a distance between adjacent peaks (e.g., adjacent peaks at adjacent anchor points 1224) or adjacent valleys (e.g., adjacent valleys at adjacent anchor points 1226) of first or second sinusoidal sutures 1209 and 1211 of collapsing suture 1208.

FIG. 12B depicts a side view of heart implant device 1200 in an expanding state (e.g., a partially deployed state) where capsule 1202 is partially retracted as shown by arrow 1228 to partially deploy heart implant device 1200. In FIG. 12B, inflow section 1230 has deployed from the delivery state to increase the nominal diameter of inflow section 1230 and decrease the length of inflow section 1230. The increased nominal diameter and the decreased length of inflow section 1230 between the compressed state and the partially deployed state reduces the amplitude and increases the period of first and second sinusoidal sutures 1209 and 1211, thereby causing collapsing suture 1208 to pull skirt 1206 downward toward inflow end 1212 and outward to outwardly flip skirt 1206 as depicted by arrows 1232 (e.g., about anchor points 1222). The downward/outward pulling force may have a longitudinal directional component and a radial directional component. In one or more embodiments, while there is some foreshortening in the longitudinal direction, the bigger effect on the suture is in the radial direction (e.g., the diameter), which pulls the suture attachment points apart. The downward/outward pulling force may form ruffles, folds, and/or pleats in skirt 1206 (e.g., FIG. 12B).

FIG. 12C depicts a side view of heart implant device 1200 in an expanded state (e.g., a fully deployed state) where capsule 1202 has been released from heart implant device 1200. In FIG. 12C, inflow section 1230 has fully deployed to further increase the nominal diameter of inflow section 1230 and decrease the length of inflow section 1230. The further increased nominal diameter and the further decreased length of inflow section 1230 between the partially deployed state and the fully deployed state further reduces the amplitude and further increases the period of first and second sinusoidal sutures 1209 and 1211, thereby causing collapsing suture 1208 to pull skirt 1206 further downward toward inflow end 1212 to increase coverage of the inflow section 1230 of skirt 1206. In the fully deployed state, a portion of inflow section 1230 and skirt 1206 overlap to form a region of increased radial thickness configured to enhance sealing of heart implant device 1200 and/or to reduce PVL.

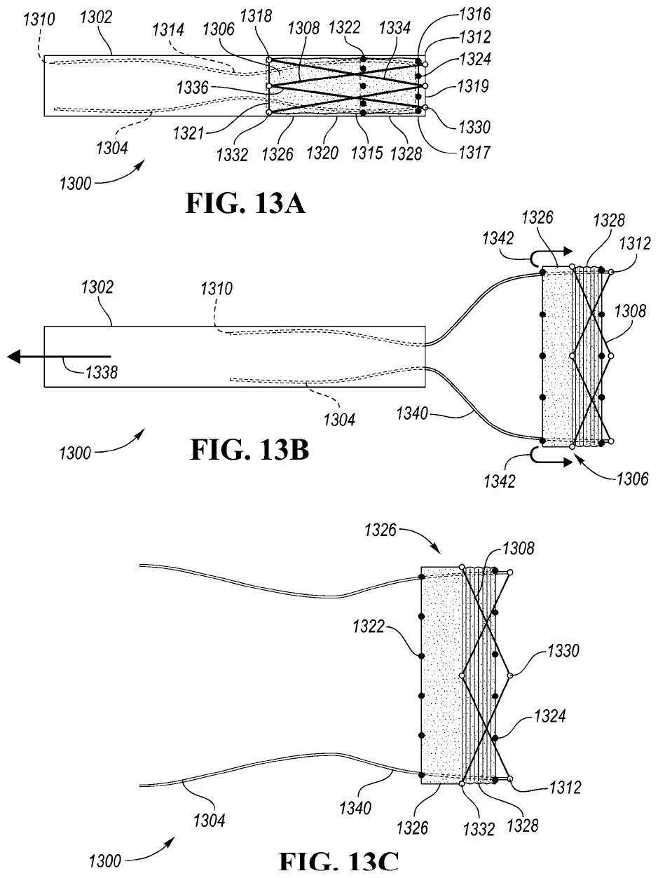

FIG. 13A depicts a side view of heart implant device 1300 in a compressed state (e.g., a delivery state). As shown in FIG. 13A, heart implant device 1300 is compressed within capsule 1302. Heart implant device 1300 includes frame 1304 (e.g., an expandable frame of a TAVR device), skirt 1306 anchored to frame 1304, and collapsing suture 1308 anchored to frame 1304 and skirt 1306. Skirt 1304 may replace skirt 1016 shown in FIG. 10A or may be an additional skirt on top of (e.g., outward) skirt 1016.

Frame 1304 includes outflow end 1310, inflow end 1312, and frame body 1314 extending therebetween. Skirt 1306 includes first end 1316, second end 1318, and skirt body 1320 extending therebetween. Skirt body 1320 may be formed of a cylindrical body (e.g., tube). Collapsing suture 1308 may be any filament or thread-like material, such as a polymer, metal, or natural material, and is not limited to surgical sutures.

A middle portion of skirt 1306 is secured to frame body 1314 via anchor points 1322. Anchor points 1322 may be located circumferentially around the circumference (e.g., the entire circumference) of frame body 1314 thereby forming circle 1315 including anchor points 1322. Anchor points 1322 may be spaced evenly apart from each other around the circumference (e.g., the entire circumference) of frame body 1314 to reinforce the connection between frame 1304 and skirt 1306.

First end 1316 of skirt 1306 is secured to frame body 1314 via anchor points 1324. Anchor points 1324 may be located circumferentially around the circumference (e.g., the entire circumference) of frame body 1314 thereby forming circle 1317 that includes anchor points 1324. Anchor points 1324 may be spaced evenly apart from each other around the circumference (e.g., the entire circumference) of frame body 1314 to reinforce the connection between frame 1304 and skirt 1306. While FIGS. 13A-13C depict two sets of anchor points (i.e., top set of anchor points 1322 and bottom set of anchor points 1324), in other embodiments, there may be three or more sets of anchor points with the additional anchor point sets situated between the top and bottom sets.

Skirt 1306 includes first section 1326 and second section 1328. First section 1326 extends between second end 1318 and first circle 1315. First section 1326 of skirt 1306 has a free end at second end 1318 relative to frame 1304, thereby permitting the end to move toward second section 1328. Second section 1328 extends between circle 1317 at first end 1316 and first circle 1315 and is fixed at circles 1315 and 1317 thereby forming a fixed portion of skirt 1306. A portion of skirt body 1320 occupies a reduced diameter portion of frame body 1314 to reduce the packing density of heart implant device 1300 in the compressed state.

Collapsing suture 1308 is secured to inflow end 1312 of frame 1304 at anchor points 1330 and is secured to second end 1318 of skirt 1306 at anchor points 1332 in a periodic, alternating repeating pattern. As shown in FIG. 13A, anchor points 1330 and anchor points 1332 secure collapsing suture 1308 to an outside surface of frame 1304 and an outside surface of skirt 1306, respectively, such that collapsing suture 1308 is located outward skirt 1306.

Anchor points 1330 may be located circumferentially around the circumference (e.g., the entire circumference) of inflow end 1312 of frame 1304 to form circle 1319 including anchor points 1330. Anchor points 1332 may be located circumferentially around the circumference (e.g., the entire circumference) of second end 1318 of skirt 1306 to form circle 1321 that includes anchor points 1332.

In one or more embodiments, the collapsing suture comprises a first suture having a repeating shape and a second suture having a repeating shape. FIG. 13A schematically illustrates collapsing suture 1308 having first sinusoidal suture 1334 and second sinusoidal suture 1336 (but in other embodiments the first and second sutures may be zig-zag shaped rings that form ring shaped scissor arms). Alternating peaks and valleys of first sinusoidal suture 1334 are secured via alternating anchor points 1330 and 1332 and alternating peaks and valleys of second sinusoidal suture 1336 are secured via alternating anchor points 1332 and 1330 such that first and second sinusoidal sutures 1334 and 1336 are out of phase with each other (e.g., the peak of one aligns with the valley of the other). While FIG. 13A shows first and second sinusoidal sutures 1334 and 1336 out of phase 180 degrees, the two or more sutures may be out of phase a different number of degrees, e.g., 60 degrees or 120 degrees.

The use of two or more sutures provides a longer axial travel than a single suture having the same number of total anchor points, thereby allowing for a greater distance to be created from inflow end 1312 to attachment of skirt 1306 during loading (e.g., the delivery sate). The use of two or more sutures may also increase the strength when pulling skirt 1306 toward inflow end 1312.

First and second sinusoidal sutures 1334 and 1336 may have a delivery amplitude and a delivery period in the delivery state (in other embodiments, first and second sinusoidal sutures 1334 and 1336 may have different amplitudes and periods). The delivery amplitude may refer to a distance between inflow end 1312 of frame 1304 at anchor points 1330 and second end 1318 of skirt 1306 at second anchor points 1332. The delivery period may refer to a distance between adjacent peaks (e.g., adjacent peaks at adjacent anchor points 1330) or adjacent valleys (e.g., adjacent valleys at adjacent anchor points 1332) of the first or second sinusoidal sutures 1334 and 1336 of collapsing suture 1308.

FIG. 13B depicts a side view of heart implant device 1300 in an expanding state (e.g., a partially deployed state) where capsule 1302 is partially retracted as shown by arrow 1338 to partially deploy heart implant device 1300. In FIG. 12B, inflow section 1340 has deployed from the delivery state to increase the nominal diameter of inflow section 1340 and decrease the length of inflow section 1340. The increased nominal diameter and the decreased length of inflow section 1340 between the compressed state and the partially deployed state reduces the amplitude and increases the period of first and second sinusoidal sutures 1334 and 1336, thereby causing collapsing suture 1308 to pull first section 1326 downward toward second section 1328 by outwardly flipping it as depicted by arrows 1342 (e.g., about anchor points 1322). The downward/outward pulling force may have a longitudinal directional component and a radial directional component. As a result, there may be a longitudinal region where first section 1326 and second section 1328 overlap and have increased radial thickness (e.g., 2×). There may also be a longitudinal region towards the inflow end where there is only the second section 1328.

FIG. 13C depicts a side view of heart implant device 1300 in an expanded state (e.g., a fully deployed state) where capsule 1302 has been released from heart implant device 1300. In FIG. 13C, inflow section 1340 has fully deployed to further increase the nominal diameter of inflow section 1340 and decrease the length of inflow section 1340. The further increased nominal diameter and the further decreased length of inflow section 1340 between the partially deployed state and the fully deployed state further reduces the amplitude and further increases the period of first and second sinusoidal sutures 1334 and 1336, thereby causing collapsing suture 1308 to further pull first section 1326 downward over second section 1328. This movement of skirt 1306 is configured to help control the height of skirt repeatably while introducing body and/or diameter of the skirt 1306 in the inflow section 1340 in the fully deployed state. In the fully deployed state, a portion of inflow section 1340 and skirt 1306 overlap to form a region of increased radial thickness configured to enhance sealing of heart implant device 1300 and/or to reduce PVL.

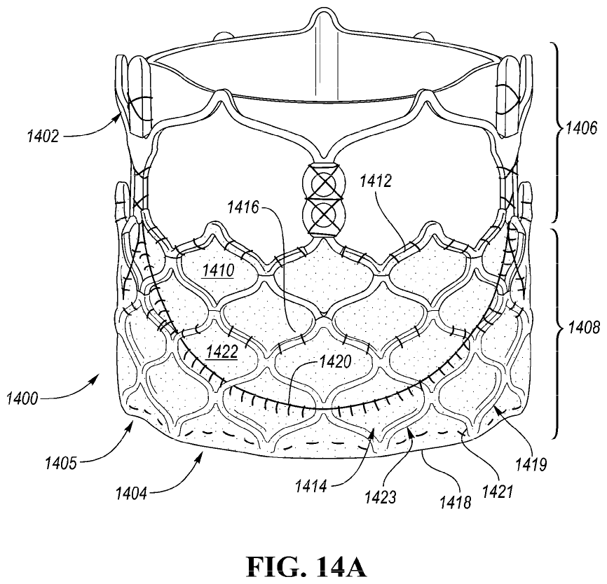

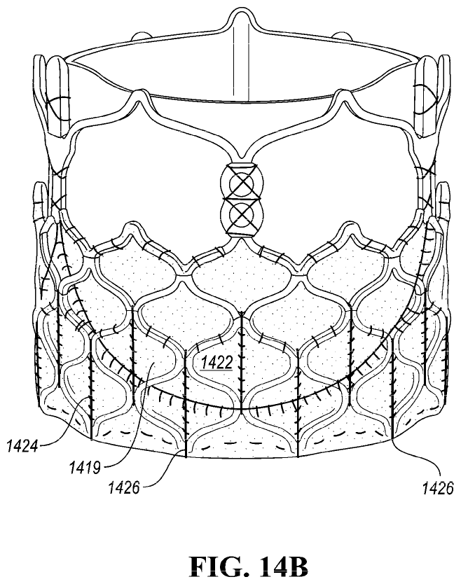

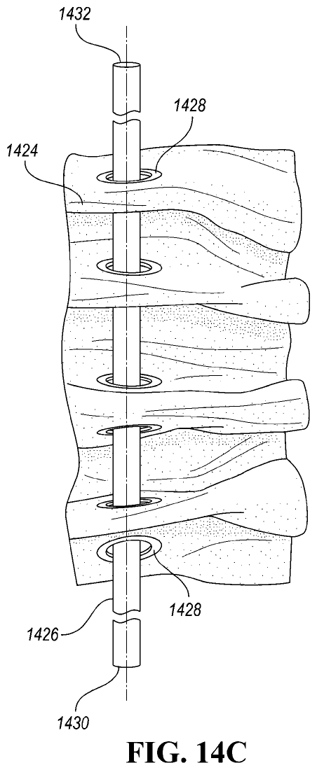

FIG. 14A depicts a schematic, side view of transcatheter aortic valve replacement (TAVR) device 1400. As shown in FIG. 14A, TAVR device 1400 includes expandable frame 1402, valve body 1404, and skirt 1405. Expandable frame 1402 may include outflow section 1406 and inflow section 1408. Expandable frame 1402 may be a balloon-expandable structure or a self-expanding structure formed by laser cutting or etching a tube formed of a metal alloy (e.g., stainless steel alloy, nickel titanium alloy, Nitinol, or other shape memory material). In other embodiments, expandable frame 1402 may be wire-formed or a woven frame. As shown in FIG. 14A, TAVR device 1400 is in an expanded, deployed state (e.g., when TAVR device 1400 is deployed at a target location).

Expandable frame 1402 includes cells 1419 formed as diamond-shaped structures and nodes 1421 formed as single points on frame 1402. Cells 1419 are connected to each other by struts 1423. The combination of four adjacent nodes and struts may form one cell.

Skirt 1405 includes inner section 1410 secured to an inner surface of expandable frame 1402 with first sutures 1412 at a first end of skirt 1405 and outer section 1414 not secured to frame 1402 to form free end 1416 at a second end of skirt 1405. Skirt 1405 may be formed of a single piece of material (e.g., synthetic material or polymeric material) wrapped around inflow end 1418 of expandable frame 1402. In one or more embodiments, skirt 1405 may be formed of an unstretchable material. Sutures 1420 following a scalloped path may be sutured onto an outer surface of outer section 1414. In one or more embodiments, sutures 1420 following a scalloped path are attached to inner skirt section 1410 (and optionally the frame as well), but not outer section 1414, to allow the entire outer section 1414 and 1422 to scrunch/fold to create an improved PVL seal. In another embodiment, outer section 1414 scrunches/pleats but intermediate section 1422 does not, which may minimize changes to an existing implant configuration.

FIG. 14B depicts a schematic, side view of TAVR device 1400 having pleated sections 1424 and extensible sutures 1426. FIG. 14C depicts an isolated, side view of pleated sections 1424 and extensible sutures 1426 of TAVR device 1400. Skirt 1405 may include intermediate portion 1422 between sutures 1420 and free end 1416 where outer section 1414 pleats but intermediate portion 1422 does not. A portion of outer section 1414 of skirt 1405 may be sutured or otherwise fastened to expandable frame 1402 to form pleated sections 1424 extending along a length of the portion of outer section 1414 when expanded frame 1402 is in the fully deployed state.

When expanded frame 1402 is loaded in a constrained position on/within a delivery system, expandable frame 1402 may be in a compressed state. Due to radial compression of expanded frame 1402 when transitioning from the fully deployed state to the compressed state, pleated sections 1424 may spread out and lie flat or substantially flat to not increase the packing density of TAVR device 1400.

Extensible sutures 1426 may extend in an axial direction along skirt 1405 and through pleated sections 1424. Extensible sutures 1426 may be formed of any filament or thread like material having an extensible, extendable, or stretchable characteristic, such as a polymer, metal, or natural material, not limited to surgical sutures. Extensible sutures 1426 may be secured to first and second ends of nodes 1421 and extend between the first and second ends. Extensible sutures 1426 may pass through pleated section 1424 two or more times. In one or more embodiments, extensible sutures 1426 may pass through pleated section 1424 any of the following numbers of times or in a range of any two of the following times: 2, 3, 4, 5, 6, 7, 8, 9, 10, 12, 14, 16, 18, and 20.

As shown in FIG. 14C, extensible suture 1426 passes through pleated section eight (8) times through eyelets 1428 defined by pleated section 1424. Eyelets 1428 may be reinforced (e.g., with a grommet) or may be an unreinforced small opening in a skirt. In other embodiments, the suture may be threaded through the skirt material without an enlarged eyelet. Extensible sutures 1426 may be connected to a node 1421 at first and second ends 1430 and 1432 of the node 1421. Extensible sutures 1426 may be configured to maintain alignment of outer section 1414 of skirt 1405 when pleated with pleated sections 1424 in the fully expanded state and when flat in the delivery state. Extensible sutures 1426 are configured to extend from a fully deployed length in the fully deployed state to a delivery length in the delivery state while remaining secured to first and second ends 1430 and 1432 of node 1421 such that the fully deployed length is less than the delivery length. Extensible sutures 1426 are configured to contract from the delivery length to the fully expanded length upon deployment of TAVR device 1400, thereby forming pleated sections 1424. Extensible sutures 1426 may be configured such that they are under tension in the delivery state but do not plastically deform when stretched. In the fully deployed state, extendable sutures 1426 may be in a neutral state (e.g., minimal slack, but no active tension). In the fully deployed state, pleated sections 1424 form a region of increased radial thickness configured to enhance sealing of TAVR device 1400 and/or reduce paravalvular leak (PVL).

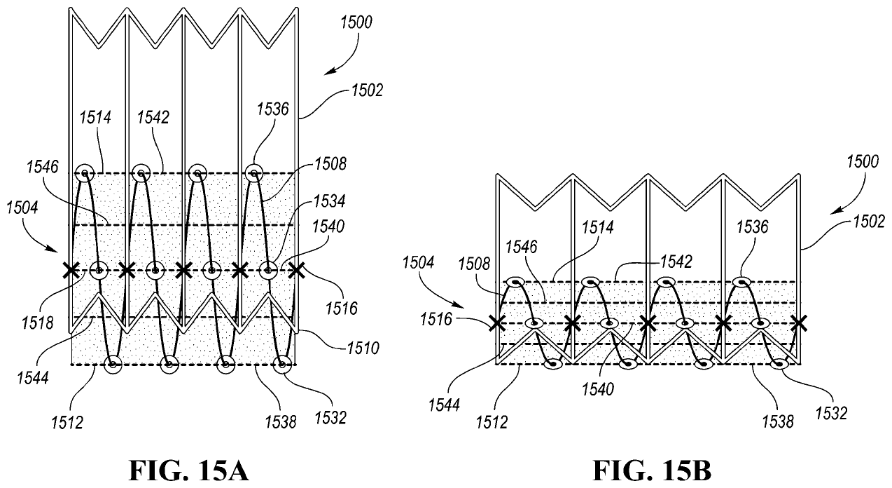

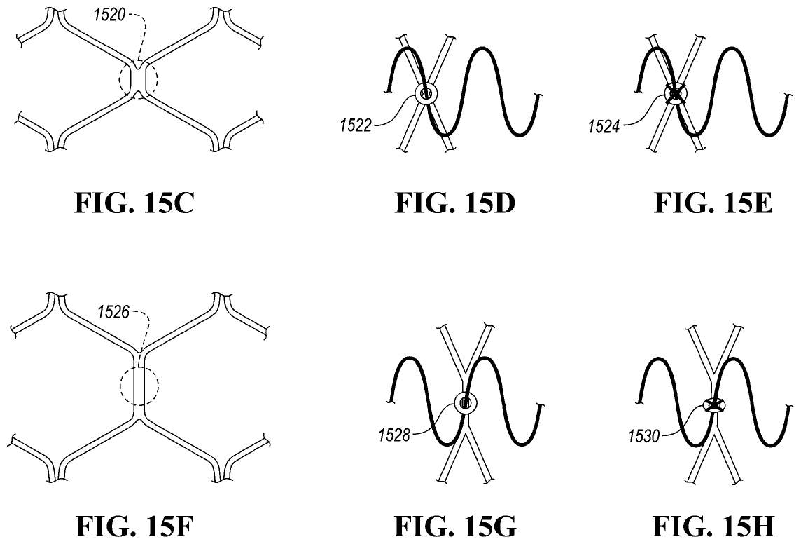

FIG. 15A depicts a side, schematic view of frame 1502 and skirt 1504 of heart implant device 1500 in a compressed state (e.g., a delivery state). FIG. 15B depicts a side, schematic view of frame 1502 and skirt 1504 of heart implant device 1500 in an expanded state (e.g., a delivered state). Heart implant device 1500 includes frame 1502 (e.g., an expandable frame of a balloon expandable TAVR device) terminating at inflow end 1510 and collapsing suture 1508 anchored to frame 1506 and skirt 1504 as described herein. Collapsing suture 1508 may be formed of any filament or thread-like material, such as a polymer, metal, or natural material, and is not limited to surgical sutures. Skirt 1604 may replace an existing skirt or may be an additional skirt on top of (e.g., outward) the skirt. Skirt 1504 includes first end 1512 and second end 1514 aligned with a middle section of frame 1502. Skirt 1504 may be formed of a cylindrical body (e.g., a tube). In at least one embodiment, first end 1512 extends beyond inflow end 1510 of the frame in the compressed state.

As shown in FIGS. 15A and 15B, collapsing suture 1508 forms a sinusoidal ring having peaks, valleys, and a central portion extending between the peaks and the valleys. In other embodiments, collapsing suture 1508 may have a zig-zag shape of alternating peaks and valleys with legs extending therebetween. Collapsing suture 1508 may be secured to frame 1502 at anchor points 1516 in a repeating pattern. Anchor points 1516 may be located circumferentially around a circumference (e.g., the entire circumference) of frame 1502 to form circle 1518 including anchor points 1516. Circle 1518 is spaced apart from inflow end 1510 of frame 1502.

FIG. 15C is a schematic view depicting frame node 1520 of frame 1502. As shown in FIG. 15D, some anchor points 1516 may connect collapsing suture 1508 to frame node 1520 through rivets 1522. As shown in FIG. 15E, anchor points 1516 may connect collapsing suture 1508 to frame node 1520 through sutures 1524. FIG. 15F is a schematic view depicting straight strut 1526 of frame 1502. As shown in FIG. 15G, some anchor points 1516 may connect collapsing suture 1508 to straight struts 1526 through rivets 1528. As shown in FIG. 15H, some anchor points 1516 may connect collapsing suture 1508 to straight struts 1526 through sutures 1530.

In one or more embodiments, skirt 1504 is secured to collapsing suture 1508 at first end 1512 of skirt 1504 at anchor points 1532, at a middle section of skirt 1504 at anchor points 1534, and at second end 1514 of skirt 1504 at anchor points 1536 in a periodic, alternating repeating pattern. Anchor points 1532, 1534, and 1536 may be configured to secure collapsing suture 1508 to the outer surface of skirt 1504. Alternatively, anchor points 1532, 1534, and 1536 may be configured to secure collapsing suture 1508 to the inner surface of skirt 1504 (e.g., such that collapsing suture is situated between frame 1502 and skirt 1504).

Anchor points 1532 may be located circumferentially around a circumference (e.g., the entire circumference) of first end 1512 of skirt 1504 to form circle 1538 including anchor points 1532. Anchor points 1534 may be located circumferentially around a circumference (e.g., the entire circumference) of a middle section of skirt 1504 to form circle 1540 including anchor points 1534. Anchor points 1536 may be located circumferentially around a circumference (e.g., the entire circumference) of second end 1514 of skirt 1504 to form circle 1542 including anchor points 1536.

The sinusoidal ring of collapsing suture 1508 may have a delivery amplitude and a delivery period in the delivery state. The delivery amplitude may refer to a distance between circle 1540 and circle 1542 or circle 1540 and circle 1538. The delivery period may refer to a distance between adjacent peaks (e.g., adjacent peaks at adjacent anchor points 1532) or adjacent valleys (e.g., adjacent valleys at adjacent anchor points 1536).

In FIG. 15B, frame 1502 has deployed to increase a nominal diameter of frame 1502 and decrease a length of frame 1502 from the delivery state. The increased nominal diameter and the decreased length of frame 1502 between the compressed state and the deployed state reduces the amplitude and increases the period of the sinusoidal ring of collapsing suture 1508, thereby causing collapsing suture 1608 to pull skirt 1504 inward in an axial direction and outward in a radial direction about anchor points 1516. In at least one embodiment, in the expanded state, skirt end 1512 may be aligned or substantially aligned with inflow end 1510 after expansion. The sinusoidal ring may be formed of a shape memory material (e.g., nitinol) configured to self-expand by shortening the amplitude of the sinusoidal ring with body temperature, with such amplitude shortening augmented by valve frame expansion into the deployment state.

Skirt 1504 may have ring stitches running around circles 1538, 1540, and 1542 as well as positions therebetween (e.g., circles 1544 and 1546). The ring stiches may be configured to stiffen portions of skirt 1504, which may facilitate pleating of the skirt at the ring stitches. The ring stitches may be formed of an elastic or inelastic thread material. In one or more embodiments, the ring stiches are not connected to frame 1506 or collapsing suture 1508 such that the shortening of the amplitude and lengthening of the period pleats skirt 1504. The pleated skirt in the deployed state forms a region of increased radial thickness configured to enhance sealing of heart implant device 1500 and/or to reduce PVL, while maintaining a lower profile in the delivery state.

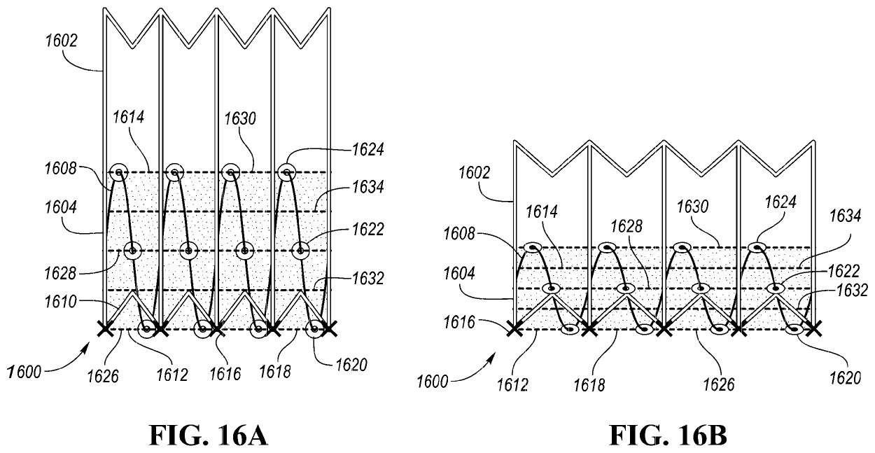

FIG. 16A depicts a side, schematic view of frame 1602 and skirt 1604 of heart implant device 1600 in a compressed state (e.g., a delivery state). FIG. 16B depicts a side, schematic view of frame 1602 and skirt 1604 of heart implant device 1600 in an expanded state (e.g., a delivered state). Heart implant device 1600 includes frame 1602 (e.g., an expandable frame of a balloon expandable TAVR device) terminating at inflow end 1610 and collapsing suture 1608 anchored to frame 1606 and skirt 1604 as described herein. Collapsing suture 1608 may be formed of any filament or thread-like material, such as a polymer, metal, or natural material, and is not limited to surgical sutures. Skirt 1604 may replace an existing skirt or may be an additional skirt on top of (e.g., outward) the skirt. Skirt 1604 includes first end 1612 and second end 1614 aligned with a middle section of inflow section 1602. Skirt 1604 may be formed of a cylindrical body (e.g., a tube).

As shown in FIGS. 16A and 16B, collapsing suture 1608 forms a sinusoidal ring having peaks, valleys, and a central portion extending between the peaks and the valleys. In other embodiments, collapsing suture 1608 may have a zig-zag shape of alternating peaks and valleys with legs extending therebetween. Collapsing suture 1608 may be secured to frame 1602 at anchor points 1616 in a repeating pattern. Anchor points 1616 may be located circumferentially around a circumference (e.g., the entire circumference) of frame 1602 to form circle 1618 including anchor points 1616. Circle 1618 may coincide with inflow end 1610 of frame 1602.

In one or more embodiments, skirt 1606 is secured to collapsing suture 1608 at first end 1612 of skirt 1604 at anchor points 1620, at a middle section of skirt 1604 at anchor points 1622, and at second end 1614 of skirt 1604 at anchor points 1624 in a periodic, alternating repeating pattern. Anchor points 1620, 1622, and 1624 may be configured to secure collapsing suture 1608 to the outer surface of skirt 1604. Alternatively, anchor points 1620, 1622, and 1624 may be configured to secure collapsing suture 1608 to the inner surface of skirt 1604 (e.g., such that collapsing suture is situated between frame 1602 and skirt 1604).

Anchor points 1620 may be located circumferentially around a circumference (e.g., the entire circumference) of first end 1612 of skirt 1604 to form circle 1626 including anchor points 1620. Anchor points 1622 may be located circumferentially around a circumference (e.g., the entire circumference) of a middle section of skirt 1604 to form circle 1628 including anchor points 1622. Anchor points 1624 may be located circumferentially around a circumference (e.g., the entire circumference) of second end 1614 of skirt 1604 to form circle 1630 including anchor points 1624.

The sinusoidal ring of collapsing suture 1608 may have a delivery amplitude and a delivery period in the delivery state. The delivery amplitude may refer to a distance between circle 1626 and circle 1628 or circle 1628 and circle 1630. The delivery period may refer to a distance between adjacent peaks (e.g., adjacent peaks at adjacent anchor points 1620) or adjacent valleys (e.g., adjacent valleys at adjacent anchor points 1624).

In FIG. 16B, frame 1602 has deployed to increase a nominal diameter of frame 1602 and decrease a length of frame 1602 from the delivery state. The increased nominal diameter and the decreased length of frame 1602 between the compressed state and the deployed state reduces the amplitude and increases the period of the sinusoidal ring of collapsing suture 1608, thereby causing collapsing suture 1608 to pull skirt 1604 inward in an axial direction and outward in a radial direction about anchor points 1616. This causes skirt 1604 to drop down toward inflow end 1610 (e.g., toward anchor points 1616). The sinusoidal ring may be formed of a shape memory material (e.g., nitinol) configured to self-expand by shortening the amplitude of the sinusoidal ring with body temperature, with such amplitude shortening augmented by valve frame expansion into the deployment state.

Skirt 1604 may have ring stitches running around circles 1626, 1628, and 1630 as well as positions therebetween (e.g., circles 1632 and 1634). The ring stiches may be configured to stiffen portions of skirt 1604, which may facilitate pleating of the skirt at the ring stitches. The ring stitches may be formed of an elastic or inelastic thread material. In one or more embodiments, the ring stiches are not connected to frame 1602 or collapsing suture 1608 such that the shortening of the amplitude and lengthening of the period pleats skirt 1604. The pleated skirt in the deployed state forms a region of increased radial thickness configured to enhance sealing of heart implant device 1600 and/or to reduce PVL, while maintaining a lower profile in the delivery state.

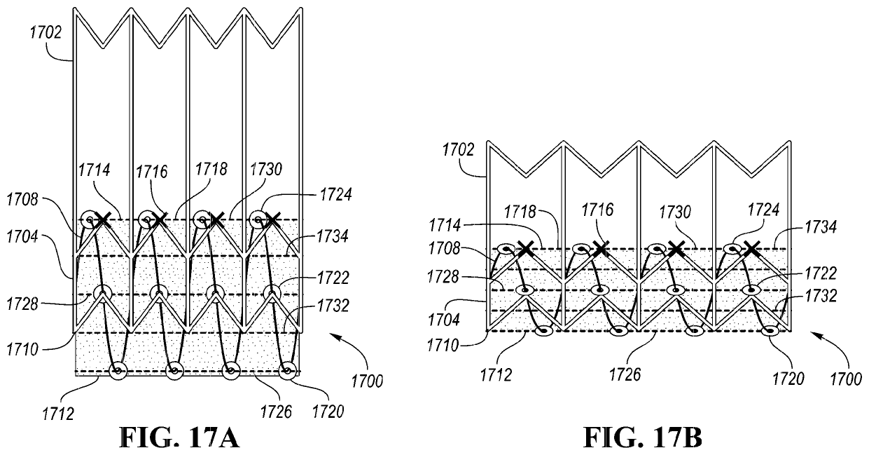

FIG. 17A depicts a side, schematic view of frame 1702 and skirt 1704 of heart implant device 1700 in a compressed state (e.g., a delivery state). FIG. 17B depicts a side, schematic view of frame 1702 and skirt 1704 of heart implant device 1700 in an expanded state (e.g., a delivered state). Heart implant device 1700 includes frame 1702 (e.g., an expandable frame of a balloon expandable TAVR device), which includes frame 1702 terminating at inflow end 1710 and collapsing suture 1708 anchored to frame 1702 and skirt 1704 as described herein. Collapsing suture 1708 may be formed of any filament or thread-like material, such as a polymer, metal, or natural material, and is not limited to surgical sutures. Skirt 1704 may replace a skirt or may be an additional skirt on top of (e.g., outward) the existing skirt. Skirt 1704 includes first end 1712 and second end 1714 aligned with a middle section of inflow section 1702. Skirt 1704 may be formed of a cylindrical body (e.g., a tube). In at least one embodiment, first end 1712 extends beyond inflow end 1710 of the frame in the compressed state.

As shown in FIGS. 17A and 17B, collapsing suture 1708 forms a sinusoidal ring having peaks, valleys, and a central portion extending between the peaks and the valleys. In other embodiments, collapsing suture 1708 may have a zig-zag shape of alternating peaks and valleys with legs extending therebetween. Collapsing suture 1708 may be secured to frame 1706 at anchor points 1716 in a repeating pattern. Anchor points 1716 may be located circumferentially around a circumference (e.g., the entire circumference) of frame 1706 to form circle 1718 including anchor points 1716. Circle 1718 may coincide with inflow end 1710 of frame 1704.

In one or more embodiments, skirt 1706 is secured to collapsing suture 1708 at first end 1712 of skirt 1704 at anchor points 1720, at a middle section of skirt 1704 at anchor points 1722, and at second end 1714 of skirt 1704 at anchor points 1724 in a periodic, alternating repeating pattern. Anchor points 1720, 1722, and 1724 may be configured to secure collapsing suture 1708 to the outer surface of skirt 1704. Alternatively, anchor points 1720, 1722, and 1724 may be configured to secure collapsing suture 1708 to the inner surface of skirt 1704 (e.g., such that collapsing suture is situated between frame 1706 and skirt 1704).

Anchor points 1720 may be located circumferentially around a circumference (e.g., the entire circumference) of first end 1712 of skirt 1704 to form circle 1726 including anchor points 1620. Anchor points 1722 may be located circumferentially around a circumference (e.g., the entire circumference) of a middle section of skirt 1704 to form circle 1728 including anchor points 1722. Anchor points 1724 may be located circumferentially around a circumference (e.g., the entire circumference) of second end 1714 of skirt 1704 to form circle 1730 including anchor points 1724.

The sinusoidal ring of collapsing suture 1708 may have a delivery amplitude and a delivery period in the delivery state. The delivery amplitude may refer to a distance between circle 1726 and circle 1728 or circle 1728 and circle 1730. The delivery period may refer to a distance between adjacent peaks (e.g., adjacent peaks at adjacent anchor points 1720) or adjacent valleys (e.g., adjacent valleys at adjacent anchor points 1724).

In FIG. 17B, frame 1702 has deployed to increase a nominal diameter of inflow section 1702 and decrease a length of frame 1702 from the delivery state. The increased nominal diameter and the decreased length of frame 1702 between the compressed state and the deployed state reduces the amplitude and increases the period of the sinusoidal ring of collapsing suture 1708, thereby causing collapsing suture 1708 to pull skirt 1704 inward in an axial direction and outward in a radial direction about anchor points 1716. This causes skirt 1704 to pull up away from inflow end 1710 (e.g., toward anchor points 1716). In at least one embodiment, in the expanded state, skirt end 1712 may be aligned or substantially aligned with inflow end 1710 after expansion. The sinusoidal ring may be formed of a shape memory material (e.g., nitinol) configured to self-expand by shortening the amplitude of the sinusoidal ring with body temperature, with such amplitude shortening augmented by valve frame expansion into the deployment state.

Skirt 1704 may have ring stitches running around circles 1726, 1728, and 1730 as well as positions therebetween (e.g., circles 1732 and 1734). The ring stiches may be configured to stiffen portions of skirt 1704, which may facilitate pleating of the skirt at the ring stitches. The ring stitches may be formed of an elastic or inelastic thread material. In one or more embodiments, the ring stiches are not connected to frame 1706 or collapsing suture 1708 such that the shortening of the amplitude and lengthening of the period pleats skirt 1704. The pleated skirt in the deployed state forms a region of increased radial thickness configured to enhance sealing of heart implant device 1700 and/or to reduce PVL, while maintaining a lower profile in the delivery state.

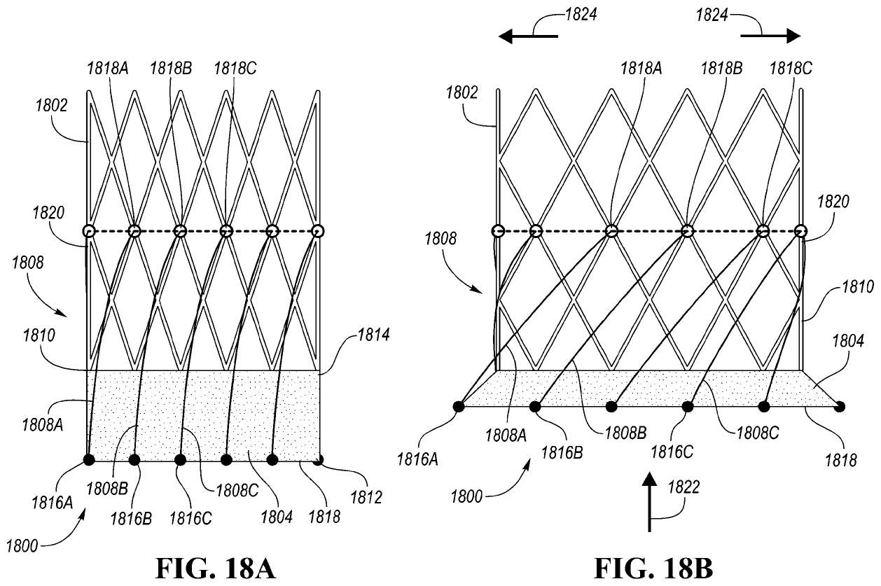

FIG. 18A depicts a side, schematic view of frame 1802 and skirt 1804 of heart implant device 1800 in a compressed state (e.g., a delivery state). FIG. 18B depicts a side, schematic view of frame 1802 and skirt 1804 of heart implant device 1800 in an expanded state (e.g., a delivered state). Heart implant device 1800 includes frame 1802 (e.g., an expandable frame of a balloon expandable TAVR device) terminating at inflow end 1810 and collapsing sutures 1808 anchored to frame 1806 and skirt 1804 as described herein. Collapsing sutures 1808 may be formed of any filament or thread-like material, such as a polymer, metal, or natural material, and is not limited to surgical sutures. Skirt 1804 may replace an existing skirt or may be an additional skirt on top of (e.g., outward) an existing skirt. Skirt 1804 includes first end 1812 and second end 1814 secured to inflow end 1810 with a suture or other fastener. The suture may be formed of any filament or thread-like material, such as a polymer, metal, or natural material, and is not limited to surgical sutures. Skirt 1804 may be formed of a cylindrical body (e.g., a tube). In at least one embodiment, the skirt first end 1812 may extend beyond the inflow end 1810 of the frame in the compressed state.

Collapsing sutures 1808 may include individual sutures spaced apart from each other around a circumference of skirt 1804 and frame 1806. In one or more embodiments, the individual sutures may be equally spaced apart from adjacent sutures. As shown in FIGS. 18A and 18B, collapsing sutures 1808 include first suture 1808A, second suture 1808B, and third suture 1808C. First suture 1808A is secured to first end 1812 of skirt 1804 at anchor point 1816A and to frame 1802 of heart implant device 1800 at anchor point 1818A. Second suture 1808B is secured to first end 1812 of skirt 1804 at anchor point 1816B and to frame 1802 of heart implant device 1800 at anchor point 1818B. Third suture 1808C is secured to first end 1812 of skirt 1804 at anchor point 1816C and to frame 1802 at anchor point 1818C.

Each of the individual sutures may have a complimentary or a congruent shape. The shape may be set using a shape set material for the sutures (e.g., nitinol). As shown in FIG. 18A, first, second, and third sutures 1808A, 1808B, and 1808C have congruent curved shapes. Anchor points 1816A, 1816B, and 1816C may secure an end of first, second, and third sutures 1808A, 1808B, and 1808C to an outer surface of skirt 1804. Anchor points 1818A, 1818B, and 1818C may secure the other end of first, second, and third sutures 1808A, 1808B, and 1808C to frame nodes or straight struts of frame 1802. Anchor points 1816A, 1816B, and 1816C may be located circumferentially around a circumference (e.g., the entire circumference) of skirt 1804 to form circle 1818 including those anchor points. Circle 1818 may coincide with first end 1812 of skirt 1804. Anchor points 1818A, 1818B, and 1818C may be located circumferentially around a circumference (e.g., the entire circumference) of frame 1802 to form circle 1820 including those anchor points.

As shown in FIGS. 18A and 18B, anchor points 1816A, 1816B, and 1816C are circumferentially offset anchor points 1818A, 1818B, and 1818C. Collapsing sutures 1808 may have a compressed offset in the delivery state. Skirt 1804 and frame 1802 may be coextensive in a compressed state to reduce the profile for delivery of heart implant device 1800.

In FIG. 18B, inflow section 1802 has deployed to increase a nominal diameter of inflow section 1802 and decrease a length of inflow section 1802 from the delivery state. The increased nominal diameter and the decreased length of inflow section 1802 between the compressed state and the deployed state increases the offset to a deployed offset, thereby causing collapsing sutures 1808 to pull skirt 1804 inward in an axial direction (see arrows 1822) and outward in a radial direction (see arrows 1824). In one or more embodiments, the individual sutures have the same length so that they move in unison with the inward axial force and outward radial force. The individual sutures may have a measure of elasticity or stretchability sufficient to facilitate the movement of the individual sutures between the delivery and deployment states while still having enough stability to provide enough force to pull up skirt 1804. The bunched skirt 1804 in the deployed state forms a region of increased radial thickness configured to enhance sealing of heart implant device 1800 and/or to reduce PVL, while maintaining a lower profile in the delivery state. The bunched skirt 1804 may be disposed at the inflow portion of the frame, for example, with skirt end 1812 aligned or substantially aligned with the inflow end 1810 of the frame.

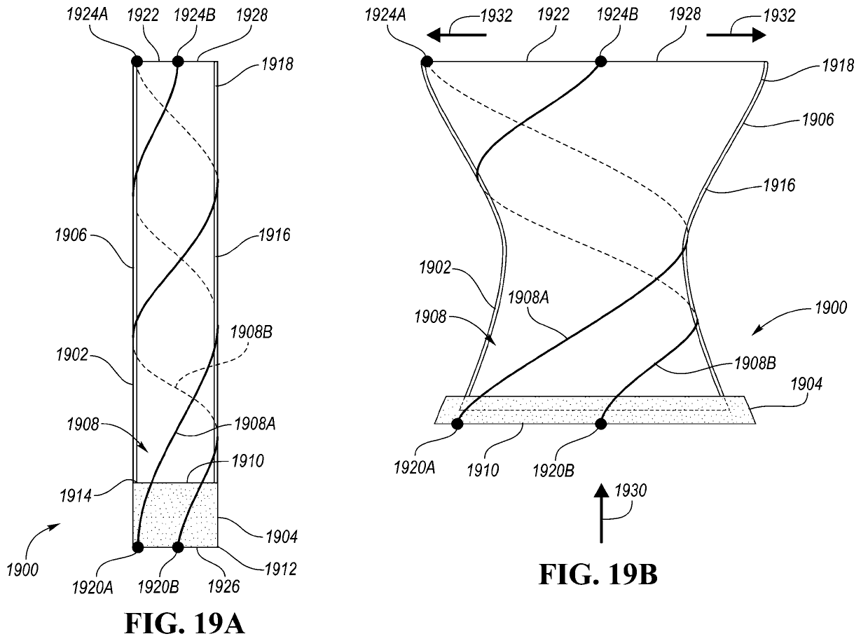

FIG. 19A depicts a side, schematic view of heart implant device 1900 in a compressed state (e.g., a delivery state). FIG. 19B depicts a side, schematic view of heart implant device 1900 in an expanded state (e.g., a delivered state). Heart implant device 1900 includes inflow section 1902. Heart implant device 1900 includes frame 1906 (e.g., an expandable frame of a TAVR device), which includes inflow section 1902 terminating at inflow end 1910 and collapsing sutures 1908 anchored to frame 1906 and skirt 1904 as described herein. Collapsing sutures 1908 may be formed of any filament or thread-like material, such as a polymer, metal, or natural material, and is not limited to surgical sutures. Skirt 1904 may replace skirt 1016 shown in FIG. 10A or may be an additional skirt on top of (e.g., outward) skirt 1016. Skirt 1904 includes first end 1912 and second end 1914 secured to inflow end 1910 with a suture or other fastener. The suture may be formed of any filament or thread-like material, such as a polymer, metal, or natural material, and is not limited to surgical sutures. Skirt 1904 may be formed of a cylindrical body (e.g., a tube). In at least one embodiment, the skirt end 1912 may extend beyond the inflow end 1910 of the frame in the compressed state.

Collapsing sutures 1908 may include individual sutures spaced apart from each other around a circumference of 1904 and frame 1906. The individually spaced apart sutures may have a spiral shape. In one or more embodiments, the individual sutures may be equally spaced apart from adjacent sutures. The individual sutures may extend from first end 1912 of skirt 1904 through inflow section 1902, constricted section 1916, and outflow section 1918 of frame 1906.

As shown in FIGS. 19A and 19B, collapsing sutures 1908 include first suture 1908A and second suture 1908B, but may also include additional sutures. First suture 1908A is secured to first end 1912 of skirt 1904 at anchor point 1920A and to outflow end 1922 of outflow section 1918 at anchor point 1924A. Second suture 1908B is secured to first end 1912 of skirt 1904 at anchor point 1920B and to outflow end 1922 of outflow section 1918 at anchor point 1924B. Anchor points 1920A and 1920B may be located circumferentially around a circumference (e.g., the entire circumference) of skirt 1904 to form circle 1926 including those anchor points. Circle 1926 may coincide with first end 1912 of skirt 1904. Anchor points 1924A and 1924B may be located circumferentially around a circumference (e.g., the entire circumference) of frame 1906 to form circle 1928 including those anchor points. Circle 1928 may coincide with outflow end 1922 of outflow section 1918.

Between their anchor points, first and second sutures 1908A and 1908B may be loosely connected to one or more nodes or straight struts of frame 1906 such that the loose connection does not interfere with first and second sutures 1908A and 1908B transitioning from the compressed state to the deployed state.

Each of the individual sutures may have a complimentary or a congruent shape. The shape may be set using a shape set material for the sutures (e.g., nitinol). As shown in FIG. 19A, first and second sutures 1908A and 1908B have congruent helical shapes.

As shown in FIG. 19A, anchor points 1920A and 1920B are circumferentially offset each other by a first distance, and anchor points 1924A and 1924B are circumferentially offset each other by the first distance. In other embodiments, the first offset distances may be different. As shown in FIG. 19B, anchor points 1920A and 1920B are circumferentially offset each other by a second distance, and anchor points 1924A and 1924B are circumferentially offset each other by the second distance. In other embodiments, the second offset distances may be different.

In FIG. 19B, heart implant device 1900 has deployed to increase a nominal diameter of frame 1906 and decrease a length of frame 1906 from the delivery state. The increased nominal diameter and the decreased length of frame 1906 between the compressed state and the deployed state increases the first and second offsets to a deployed first and second offsets, thereby causing collapsing sutures 1908 to pull skirt 1904 inward in an axial direction (see arrows 1930) and outward in a radial direction (see arrows 1932). In one or more embodiments, the individual sutures have the same length so that they move in unison with the inward axial force and outward radial force. The individual sutures may have a measure of elasticity or stretchability sufficient to facilitate the movement of the individual sutures between the delivery and deployment states while still having enough stability to provide enough force to pull up skirt 1904. As shown in FIG. 19B, skirt 1904 bunches up. The bunched skirt 1904 in the deployed state forms a region of increased radial thickness configured to enhance sealing of heart implant device 1900 and/or to reduce PVL, while maintaining a lower profile in the delivery state. The bunched skirt 1904 may be disposed at the inflow portion of the frame, for example, with skirt end 1912 aligned or substantially aligned with the inflow end 1910 of the frame.

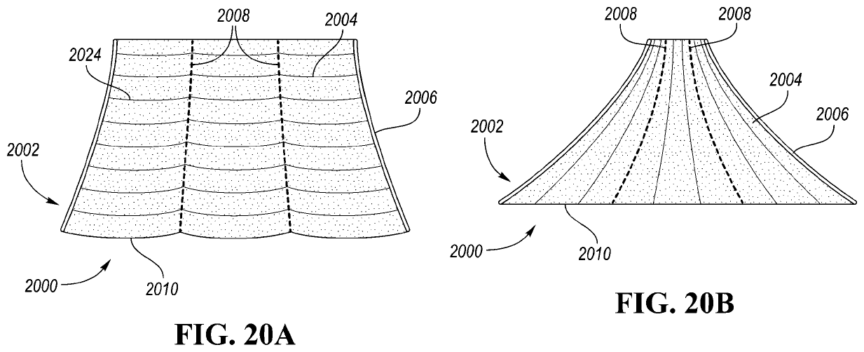

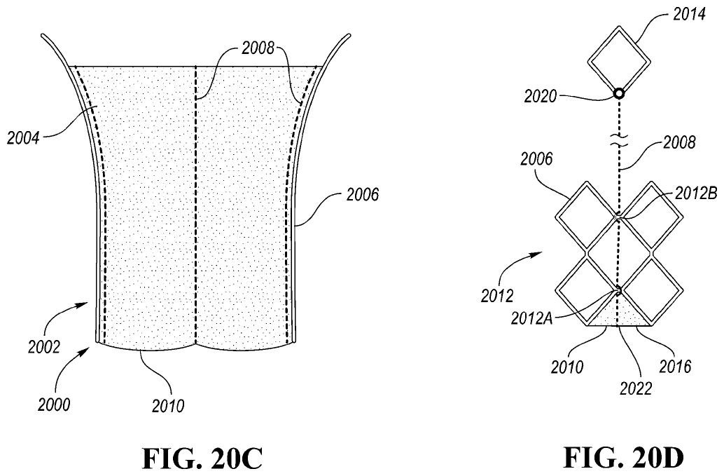

FIG. 20A depicts a schematic, side view of inflow section 2002 of heart implant device 2000 in a fully deployed state. FIG. 20B depicts a schematic, side view of inflow section 2002 of heart implant device 2000 in a partially deployed state. FIG. 20C depicts a schematic, side view of inflow section 2002 of heart implant device 2000 in a compressed state (e.g., a delivery state). Heart implant device 2000 includes skirt 2004 and frame 2006. Inflow section 2002 terminates at inflow end 2010.

Heart implant device 2000 includes extensible sutures 2008. Extensible sutures 2008 may extend in an axial direction along frame 2006. Extensible sutures 2008 may be formed of any filament or thread like material having an extensible, extendable, or stretchable characteristic, such as a polymer, metal, or natural material, not limited to surgical sutures. FIG. 20D depicts a schematic view of extensible suture 2008 cross weaved axially relative to frame nodes 2012 of frame 2006. As shown in FIG. 20D, extensible suture 2008 is woven over frame node 2012A and woven under frame node 2012B. This weaving pattern (i.e., one over and one under) may be repeated as extensible suture 2008 extends to, for example, commissure cell 2014 of frame 2006. Other weaving patterns may be utilized (e.g., two over and two under). Second end of extensible sutures 2008 is anchored to commissure cell 2014 through anchor point 2020. First end may be a free end that is not fixed to the frame but is attached to the skirt. Extensible sutures 2008 may be positioned cells apart from each (e.g., 2, 3, 4, or 5 cells apart). If each extensible suture 2008 is positioned 3 cells apart, each extensible suture 2008 is connected to a second cell with the skirt ending in a third node (e.g., at skirt edge 2022 shown in FIG. 20D).

Extensible sutures 2008 may pass through pleated section 2024 (e.g., shown in FIG. 20A) two or more times. In one or more embodiments, extensible sutures 2008 may pass through pleated section 1424 any of the following numbers of times or in a range of any two of the following times: 2, 3, 4, 5, 6, 7, 8, 9, 10, 12, 14, 16, 18, and 20. When frame 2006 is loaded in a constrained position within a delivery system (e.g., as depicted in FIG. 20C), frame 2006 is compressively retained within a capsule (e.g., capsule 1056), which may be referred to as a compressed state. Due to radial compression of frame 2006 when transitioning from the fully deployed state to the partially deployed state (e.g., as shown in FIG. 20B) to the compressed state (e.g., as shown in FIG. 20A), pleated sections 2024 may spread out and lie flat or substantially flat to not increase the packing density of heart implant device 2000.

Extensible sutures 2008 may be configured to maintain alignment of skirt 2004 when pleated with pleated sections 2024 in the fully expanded state and when flat in the compressed state. Extensible sutures 2008 are configured to extend from a fully deployed length in the fully deployed state to a delivery length in the compressed state while remaining secured to anchor point 2020 and loosely connected to frame nodes (e.g., frame nodes 2012A and 2012B) (e.g., extensible sutures 2008 do not observe any tension) such that the fully deployed length is less than the compressed length. Extensible sutures 2008 are configured to contract from the delivery length to the fully expanded length upon deployment of heart implant device 2000, thereby forming pleated sections 2024, which expand outwards as shown in FIG. 20A. Extensible sutures 2008 may be configured such that they are under tension (but do not plastically deform when stretched) in the fully expanded state by the outflow section (not shown). In the fully deployed state, pleated sections 2024 form a region of increased radial thickness configured to enhance sealing of heart implant device 2000 and/or reduce paravalvular leak (PVL).

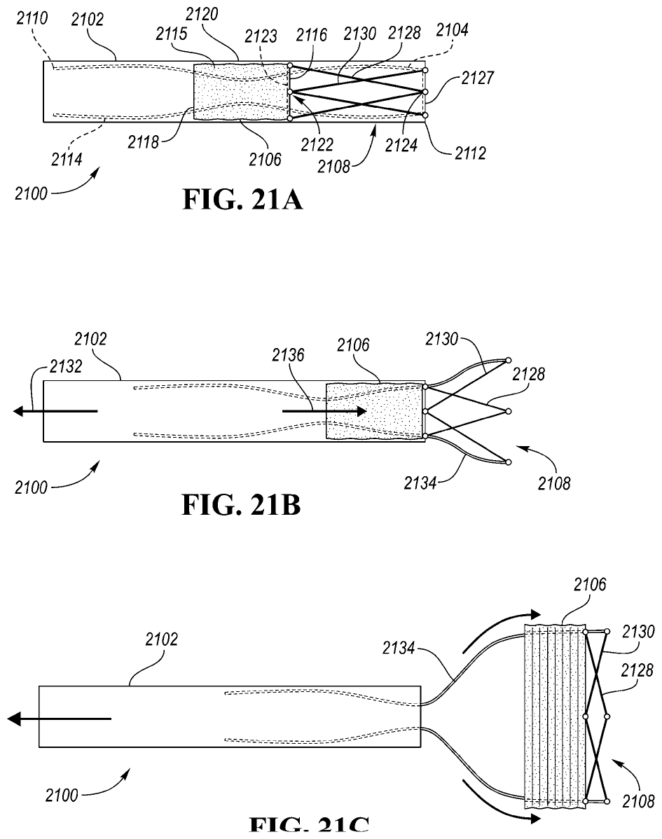

FIG. 21A depicts a side view of heart implant device 2100 in a compressed state (e.g., a delivery state). As shown in FIG. 21A, heart implant device 2100 is compressed within capsule 2102. Heart implant device 2100 includes frame 2104 (e.g., an expandable frame of a TAVR device), skirt 2106 anchored to frame 2104, and collapsing suture 2108 anchored to frame 2104 and skirt 2106. Collapsing suture 2108 may be formed of any filament or thread-like material, such as a polymer, metal, or natural material, and is not limited to surgical sutures. Skirt 2106 may replace an existing skirt shown in FIG. 10A or may be an additional skirt on top of (e.g., outward) the existing skirt.

Frame 2104 includes outflow end 2110, inflow end 2112, and frame body 2114 extending therebetween. Frame 2104 also includes constricted section 2115, which may include commissure posts. Skirt 2106 includes first end 2116, second end 2118, and skirt body 2120 extending therebetween. Skirt body 2120 may be formed of a cylindrical body (e.g., a tube).

First end 2116 of skirt 2106 is secured to collapsing suture 2108 via anchor points 2122 in a repeating pattern. Anchor points 2122 may include sutures securing skirt 2106 and collapsing suture 2108 together. Anchor points 2122 may be located circumferentially around a circumference (e.g., the entire circumference) of skirt 2106 to form circle 2123 at first end 2116 of skirt 2106 including anchor points 2122. Anchor points 2122 may be spaced evenly apart from each other around a circumference (e.g., the entire circumference) of skirt 2106 to reinforce the connection between frame 2104 and skirt 2106.

As shown in FIG. 21A, skirt body 2120 extends away from circle 2123 to axially coincide with constricted portion 2115 of frame 2104 thereby occupying a reduced diameter portion of frame body 2114 such that skirt 2106 does not impact or only negligibly impacts the packing density of heart implant device 2100 in the compressed state within capsule 2102.

Collapsing suture 2108 is secured to inflow end 2112 of frame 2104 at anchor points 2124. Anchor points 2124 may be located circumferentially around a circumference (e.g., the entire circumference) of inflow end 2112 of frame 2104 to form circle 2127 including anchor points 2124.

FIG. 21A schematically illustrates collapsing suture 2108 having first sinusoidal suture 2128 and second sinusoidal suture 2130 (but in other embodiments the first and second sutures may be zig-zag shaped rings that form ring shaped scissor arms). Alternating peaks and valleys of first sinusoidal suture 2128 are secured to frame 2104 via alternating anchor points 2122 and 2124 and alternating peaks and valleys of second sinusoidal suture 2130 are secured via alternating anchor points 2124 and 2122 such that first and second sinusoidal sutures 2128 and 2130 are out of phase with each other (e.g., the peak of one aligns with the valley of the other).

First and second sinusoidal sutures 2128 and 2130 may have a delivery amplitude and a delivery period in the delivery state (in other embodiments, first and second sinusoidal sutures 2128 and 2130 may have different amplitudes and periods). The delivery amplitude may refer to a distance between inflow end 2112 of frame 2104 at anchor points 2124 and first end 2116 of skirt 2106 at second anchor points 2122. The delivery period may refer to a distance between adjacent peaks (e.g., adjacent peaks at adjacent anchor points 2122) or adjacent valleys (e.g., adjacent valleys at adjacent anchor points 2124) of first or second sinusoidal sutures 2128 and 2130 of collapsing suture 2108.

FIG. 21B depicts a side view of inflow section 2134 of heart implant device 2100 in an expanding state (e.g., a partially deployed state) where capsule 2102 is partially retracted as shown by arrow 2132 to partially deploy heart implant device 2100. In FIG. 21B, inflow section 2134 has deployed from the delivery state to increase the nominal diameter of inflow section 2134 and decrease the length of inflow section 2134. The increased nominal diameter and the decreased length of inflow section 2134 between the compressed state and the partially deployed state reduces the amplitude and increases the period of first or second sinusoidal sutures 2128 and 2130, thereby causing collapsing suture 2108 to pull skirt 2106 downward toward inflow end 2112 as depicted by arrow 2136 (e.g., by anchor points 2122).

FIG. 21C depicts a side view of inflow section 2134 of heart implant device 2100 in an expanding state (e.g., a fully deployed state) where capsule 2102 has been released from the inflow section of the heart implant device 2100. In FIG. 21C, inflow section 2134 has fully deployed to further increase the nominal diameter of inflow section 2134 and decrease the length of inflow section 2134. The further increased nominal diameter and the further decreased length of inflow section 2134 between the partially deployed state and the fully deployed state further reduces the amplitude and further increases the period of first or second sinusoidal sutures 2128 and 2130, thereby causing collapsing suture 2108 to pull skirt 2106 further downward and outward toward inflow end 2112 to increase the circumference of skirt 2106. The downward/outward pulling force may have a longitudinal directional component and a radial directional component. In the fully deployed state of inflow section 2134, a portion of inflow section 2134 and skirt 2106 overlap to form a region of increased radial thickness configured to enhance sealing of heart implant device 2100 and/or to reduce PVL.

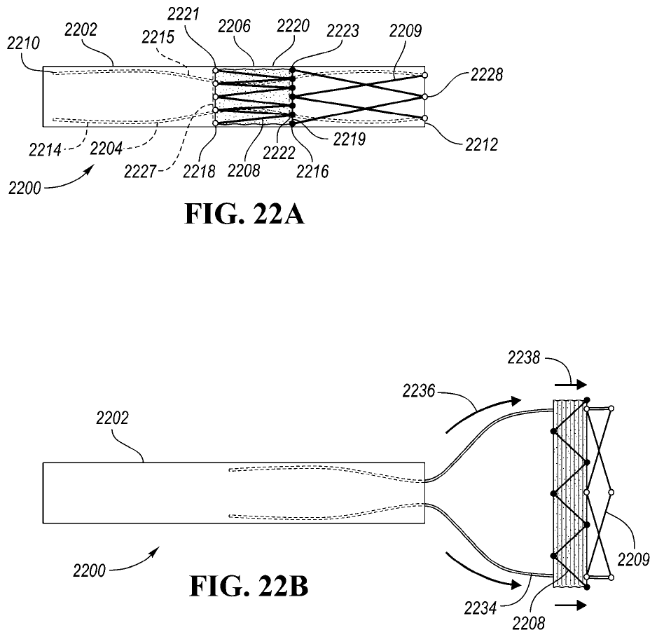

FIG. 22A depicts a side view of heart implant device 2200 in a compressed state. As shown in FIG. 22A, heart implant device 2200 is compressed within capsule 2202. Heart implant device 2200 includes frame 2204, skirt 2206 anchored to frame 2204, collapsing suture 2209 anchored to frame 2204 and skirt 2206, and collapsing suture 2208 anchored to skirt 2206. Collapsing sutures 2208 and 2209 may be formed of any filament or thread-like material, such as a polymer, metal, or natural material, and is not limited to surgical sutures. Skirt 2206 may replace an existing skirt shown in FIG. 10A or may be an additional skirt on top of (e.g., outward) an existing skirt.

Frame 2204 includes outflow end 2210, inflow end 2212, and frame body 2214 extending therebetween. Frame 2204 also includes constricted section 2215, which may include commissure posts. Skirt 2206 includes first end 2216, second end 2218, and skirt body 2220 extending therebetween. Skirt body 2220 may be formed of a cylindrical body (e.g., a tube).

Collapsing suture 2208 is secured to first end 2216 of skirt 2206 via anchor points 2219 in a repeating pattern and to second end 2218 of skirt 2206 via anchor points 2221 in a repeating pattern. Anchor points 2219 may be located circumferentially around a circumference (e.g., the entire circumference) of skirt 2206 to form circle 2223 at first end 2216 of skirt 2206 including anchor points 2219. Anchor points 2221 may be located circumferentially around a circumference (e.g., the entire circumference) of skirt 2206 to form circle 2227 at second end 2218 of skirt 2206 including anchor points 2221. FIG. 22A schematically illustrates collapsing suture 2208 having first sinusoidal suture and second sinusoidal suture (but in other embodiments the first and second sutures may be zig-zag shaped rings that form ring shaped scissor arms).

First end 2216 of skirt 2206 is secured to collapsing suture 2209 via anchor points 2222 in a repeating pattern. Anchor points 2222 may include sutures securing skirt 2206 and collapsing suture 2209 together. Anchor points 2222 may be located circumferentially around a circumference (e.g., the entire circumference) of skirt 2206 around circle 2223 at first end 2216 of skirt 2206 including anchor points 2219 and 2222. Anchor points 2219 and/or 2122 may be spaced evenly apart from each other around a circumference (e.g., the entire circumference) of skirt 2206 to reinforce the connection between frame 2204 and skirt 2206. Collapsing suture 2209 is secured to inflow end 2212 of frame 2204 at anchor points 2228. Anchor points 2228 may be located circumferentially around a circumference (e.g., the entire circumference) of inflow end 2112 of frame 2104 to form a circle including anchor points 2228.

As shown in FIG. 22A, skirt body 2220 extends away from circle 2223 to axially coincide with constricted portion 2215 of frame 2204 thereby occupying a reduced diameter portion of frame body 2214 such that skirt 2206 does not impact or only negligibly impacts the packing density of heart implant device 2200 in the compressed state within capsule 2202.

FIG. 22A schematically illustrates collapsing suture 2209 having first sinusoidal suture and second sinusoidal suture (but in other embodiments the first and second sutures may be zig-zag shaped rings that form ring shaped scissor arms). Alternating peaks and valleys of first sinusoidal suture are secured to frame 2204 via alternating anchor points 2222 and 2228 and alternating peaks and valleys of second sinusoidal suture are secured via alternating anchor points 2222 and 2226 such that first and second sinusoidal sutures are out of phase with each other (e.g., the peak of one aligns with the valley of the other).

First and second sinusoidal sutures may have a delivery amplitude and a delivery period in the delivery state (in other embodiments, first and second sinusoidal sutures may have different amplitudes and periods). The delivery amplitude may refer to a distance between inflow end 2212 of frame 2204 at anchor points 2228 and first end 2216 of skirt 2206 at second anchor points 2222. The delivery period may refer to a distance between adjacent peaks (e.g., adjacent peaks at adjacent anchor points 2222) or adjacent valleys (e.g., adjacent valleys at adjacent anchor points 2228) of first or second sinusoidal sutures of collapsing suture 2209.

FIG. 22B depicts a side view of inflow section 2234 of heart implant device 2200 in an expanded state (e.g., a fully deployed state) where capsule 2202 has been released from inflow section 2234 of heart implant device 2200. Inflow section 2234 has fully deployed to increase the nominal diameter of inflow section 2234 and decrease the length of inflow section 2234. The increased nominal diameter and the decreased length of inflow section 2234 reduces the amplitude and further increases the period of first or second sinusoidal sutures of collapsing sutures 2208 and 2209, thereby causing collapsing suture 2209 to pull skirt 2206 downward and outward toward inflow end 2212 to increase the circumference of skirt 2206 and causing collapsing suture 2208 to pleat or ruffle skirt 2206 (e.g., as depicted by arrows 2238). The downward/outward pulling force (e.g., as depicted by arrows 2236) may have a longitudinal directional component and a radial directional component. In the fully deployed state of inflow section 2234, a portion of inflow section 2234 and skirt 2206 overlap to form a region of increased radial thickness configured to enhance sealing of heart implant device 2200 and/or to reduce PVL.

The suture pattern from the frame may extend to the top of the skirt and/or there could be two separate suture patterns, where an alternative pattern may be included solely in the skirt to aid with pleating by increasing the density pattern.

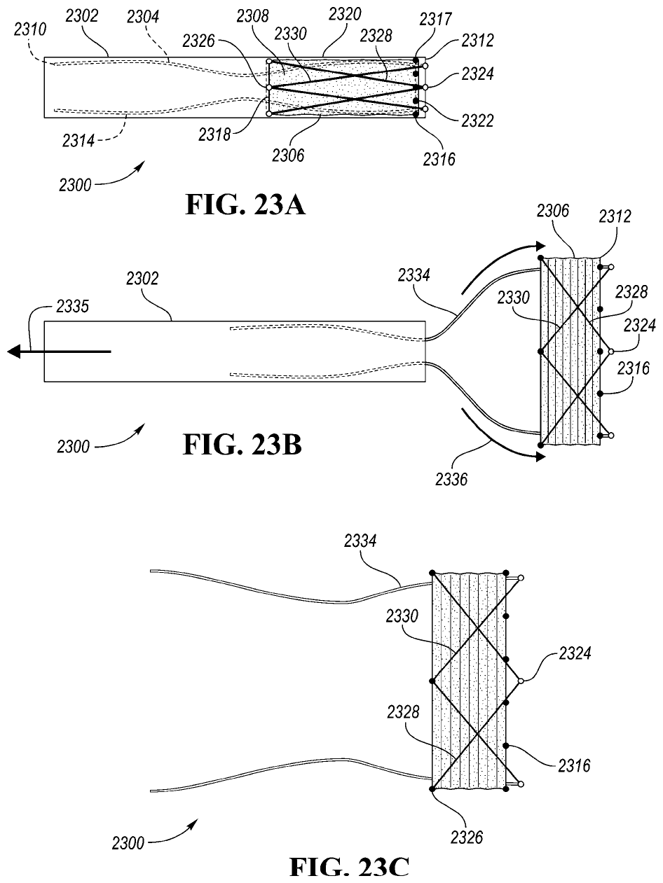

FIG. 23A depicts a side view of heart implant device 2300 compressed within capsule 2302. Heart implant device 2300 includes frame 2304, skirt 2306 anchored to frame 2304, and collapsing suture 2308 anchored to frame 2304 and skirt 2306. Collapsing suture 2308 may be formed of any filament or thread-like material, such as a polymer, metal, or natural material, and is not limited to surgical sutures. Skirt 2306 may replace skirt 1016 shown in FIG. 10A or may be an additional skirt on top of (e.g., outward) skirt 1016. Frame 2304 includes outflow end 2310, inflow end 2312, and frame body 2314 extending therebetween. Skirt 2306 includes first end 2316, second end 2318, and skirt body 2320 extending therebetween. Skirt body 2320 may be formed of a cylindrical body (e.g., a tube).

First end 2316 of skirt 2306 is secured to frame body 2314 via anchor points 2322. Anchor points 2322 may be located circumferentially around the circumference (e.g., the entire circumference) of frame body 2314 thereby forming circle 2317 that includes anchor points 2322. Anchor points 2322 may be spaced evenly apart from each other around the circumference (e.g., the entire circumference) of frame body 2314 to reinforce the connection between frame 2304 and skirt 2306.

Collapsing suture 2308 is secured to inflow end 2312 of frame 2304 at anchor points 2324 and is secured to second end 2318 of skirt 2306 at anchor points 2326 in a periodic, alternating repeating pattern. As shown in FIG. 23A, anchor points 2324 and anchor points 2326 secure collapsing suture 2308 to an outside surface of frame 2304 and an outside surface of skirt 2306, respectively, such that collapsing suture 2308 is located outward skirt 2306. Alternatively, the anchor points 2324 and anchor points 2326 may secure collapsing suture 2308 to an outside surface of frame 2304 and an inside surface of skirt 2306, respectively, such that collapsing suture 2308 is located inward to the skirt 2306.

FIG. 23A schematically illustrates collapsing suture 2308 having first sinusoidal suture 2328 and second sinusoidal suture 2330 (but in other embodiments the first and second sutures may be zig-zag shaped rings that form ring shaped scissor arms). Alternating peaks and valleys of first sinusoidal suture 2328 are secured to frame 2304 via alternating anchor points 2324 and 2326 and alternating peaks and valleys of second sinusoidal suture 2330 are secured via alternating anchor points 2326 and 2324 such that first and second sinusoidal sutures 2328 and 2330 are out of phase with each other (e.g., the peak of one aligns with the valley of the other).

First and second sinusoidal sutures 2328 and 2330 may have a delivery amplitude and a delivery period in the delivery state (in other embodiments, first and second sinusoidal sutures 2328 and 2330 may have different amplitudes and periods). The delivery amplitude may refer to a distance between inflow end 2312 of frame 2304 at anchor points 2324 and second end 2318 of skirt 2306 at second anchor points 2326. The delivery period may refer to a distance between adjacent peaks (e.g., adjacent peaks at adjacent anchor points 2324) or adjacent valleys (e.g., adjacent valleys at adjacent anchor points 2326) of first or second sinusoidal sutures 2328 and 2330 of collapsing suture 2308.

FIG. 23B depicts a side view of inflow section 2334 of heart implant device 2300 in an expanding state (e.g., a partially deployed state) where capsule 2302 is partially retracted as shown by arrow 2335 to partially deploy heart implant device 2300. Inflow section 2334 has deployed from the delivery state to increase the nominal diameter of inflow section 2334 and decrease the length of inflow section 2334. The increased nominal diameter and the decreased length of inflow section 2334 between the compressed state and the partially deployed state reduces the amplitude and increases the period of first or second sinusoidal sutures 2328 and 2330, thereby causing collapsing suture 2308 to pull skirt 2306 downward and outward toward inflow end 2312 as depicted by arrow 2336 (e.g., by anchor points 2316), thereby causing skirt 2306 to ruffle or pleat. The downward/outward pulling force may have a longitudinal directional component and a radial directional component. In the fully deployed state of inflow section 2334, a portion of inflow section 2334 and skirt 2306 overlap to form a region of increased radial thickness configured to enhance sealing of heart implant device 2300 and/or to reduce PVL. FIG. 23C depicts a side view of heart implant device 2300 in an expanded state (e.g., a fully deployed state) where capsule 2302 has been released from heart implant device 2300.

Embodiments shown above with one sinusoidal suture may have two or more and those shown with two may have one more than two. Sutures disclosed in any embodiment herein may be elastic or inelastic sutures.

The detailed description set forth herein includes several embodiments where each of the embodiments may include several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any type of collapsing suture disclosed herein may be used in conjunction with any disclosed embodiments. As another example, any spacing of anchor points disclosed herein may be used in conjunction with any disclosed embodiments.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An assembly comprising:
a graft material comprising an edge;
a stent coupled to the graft material at anchor points, the graft material comprising a sealing skirt between the anchor points and the edge; and
a collapsing suture coupled to the sealing skirt and to the graft material at periodic points in an alternating repeating pattern.

2. The assembly of claim 1, wherein the periodic points comprise first periodic points that define a first suture line and second periodic points that define a second suture line.

3. The assembly of claim 2, wherein upon expansion of the stent, a longitudinal distance between the first suture line and the second suture line decreases.

4. The assembly of claim 3, wherein upon the expansion of the stent, a circumferential distance between the first suture points increases and a circumferential distance between the second suture points increases.

5. The assembly of claim 1, wherein upon expansion of the stent, the collapsing suture collapses the sealing skirt.

6. The assembly of claim 5, wherein the sealing skirt protrudes radially outward when collapsed.

7. The assembly of claim 6, further comprising a sealing stent graft comprising the graft material, the stent, and the collapsing suture and a first component, the sealing skirt being located within the first component.

8. The assembly of claim 5, wherein the sealing skirt protrudes radially inward when collapsed.

9. The assembly of claim 8 further comprising a sealing stent graft comprising the graft material, the stent, and the collapsing suture and a first component located within the sealing skirt.

10. The assembly of claim 1, wherein the sealing skirt lays flat upon the stent prior to expansion of the stent.

11. An assembly comprising:
a sealing stent graft comprising:
a graft material;
a stent coupled to the graft material, and
a collapsing suture coupled to the graft material,
wherein when the sealing stent graft is in a delivery configuration, a sealing skirt of the graft material lies flat on the stent, and
wherein when the sealing stent graft is in a deployed configuration, the sealing skirt is collapsed by the collapsing suture and protrudes from the stent.

12. The assembly of claim 11, wherein the graft material further comprises an exclusion section.

13. The assembly of claim 12, wherein the stent comprises first crowns, second crowns, and struts extending between the first crowns and the second crowns, the second crowns being attached to the graft material at anchor points, the sealing skirt and the exclusion section meeting at the anchor points.

14. The assembly of claim 13, wherein the graft material comprises an edge defining an opening, the edge overlapping the struts.

15. The assembly of claim 13, wherein the stent extends from the second crowns past the edge to the first crowns.

16. A method comprising:
providing a sealing stent graft comprising: a graft material, a stent coupled to the graft material, a sealing skirt of the graft material lying flat on the stent, and a collapsing suture coupled to the graft material; and
expanding the stent to cause the collapsing suture to collapse the sealing skirt.

17. The method of claim 16, wherein the collapse of the sealing skirt creates ruffles in the sealing skirt.

18. The method of claim 16 further comprising:
positioning the sealing stent graft in a vessel prior to the expanding step; and
deploying the sealing stent graft in the vessel.

19. The method of claim 18, wherein the sealing skirt creates a seal with the vessel.

20. The method of claim 16, further comprising creating a seal with the sealing skirt between the sealing stent graft and a first component.
